(19) United States
(12) Patent Application Publication
(10) Pub. No.: US 2026/0335602 A1
(43) Pub. Date: Oct. 29, 2026

(54) SECUREMENT ASSEMBLIES FOR CATHETER SITES

(71) Applicant: Becton, Dickinson and Company, Franklin Lakes, NJ (US)

(72) Inventors: Praveen Nalawade, Bengaluru (IN); Shishir Prasad, Ramsey, NJ (US); Mohammad Safi, Bengaluru (IN); Soon Yong Park, Cypress, CA (US); Adel Shams Kopaye, Verona, NJ (US); Eva Bica-Winterling, Mountain Lakes, NJ (US); Kyle Todd De Boer, Midland Park, NJ (US); Jennifer Hooper, Oviedo, FL (US); Fanqing Meng, Buffalo Grove, IL (US); Shashwat Jain, Indore (IN); Todd L. Schwendiman, South Jordan, UT (US); Homprakash Hariharan, Bangalore (IN); Jayakumar Jayabal, Bangalore (IN)

(21) Appl. No.: 19/655,026

(22) Filed: Apr. 22, 2026

(30) Foreign Application Priority Data
Apr. 25, 2025 (IN) 202511039862

(51) Int. Cl. A61M 25/02

(52) U.S. Cl. CPC A61M 25/02; A61M 2025/024; A61M 2025/028; A61M 2025/0286; A61M 2205/0216; A61M 2209/088; A61M 2210/04

(57) ABSTRACT

A catheter securement device includes a base having first and second sections and an elastomeric member positioned between and secured to the first and second sections. The first and second sections are co-planar when the elastomeric member is in a relaxed state. First and second hinge members are secured to front edges of the first and second sections and each includes an arm extending outward therefrom. A distal end of each arm includes a laterally extending prong. When the elastomeric member is folded to an unrelaxed state, the prongs are in adjacent relationship and each extends in substantially a same direction to facilitate insertion and removal through an incision in the skin of a patient. Upon insertion through the incision and subsequently allowing the elastomeric member to return to the relaxed state, the prongs are rotated so as to become subcutaneously anchored in the skin of the patient.

### RELATED APPLICATION

This application claims the benefit of and priority to Indian Provisional Patent Application No. 202511039862, filed Apr. 25, 2025, the disclosure of which is incorporated herein by reference as if set forth in its entirety.

### FIELD OF THE INVENTION

The present invention is directed generally to medical catheter accessories, and more specifically to devices for aiding in the securing of catheters.

### BACKGROUND OF THE INVENTION

Catheters are medical devices that can be inserted in the body to treat diseases or perform a surgical procedure. Catheters are manufactured for specific applications, such as cardiovascular, urological, gastrointestinal, neurovascular, and ophthalmic procedures. In most uses, a catheter is a thin, flexible tube (soft catheter) though catheters are available in varying levels of stiffness depending on the application. Catheters can be inserted into a body cavity, duct, or vessel, brain, skin, or adipose tissue. Functionally, they allow drainage, administration of fluids or gases, access by surgical instruments, and perform a wide variety of other tasks depending on the type of catheter.

When a catheter is left in place in a patient long-term, often a securement technique is employed to secure the catheter in its location. This is particularly true for central venous catheters (CVCs) and peripherally inserted central catheters (PICCs), which are often inserted into the superior vena cava via the patient's neck/chest and upper arm, respectively. Solutions include sutures and catheter securement devices (an exemplary catheter securement device is the STATLOCK™ device, available from Becton, Dickinson and Co. (Franklin Lakes, New Jersey)). With either of these techniques, typically a dressing associated with the catheter insertion site requires changing. Cleaning around a catheter that is secured by sutures results in incomplete (and therefore inadequate) cleaning, which in turn can increase the risk of central line-associated bloodstream infection (CLABSI). When the catheter is secured with a securement device, typically the securement device is changed one or more times during the period that the catheter is in place (e.g., sometimes securement devices are changed whenever the dressing at the catheter site is changed). However, it may be desirable under some circumstances for the securement device to remain with the catheter for the full life of the catheter.

In particular, it may be desirable to provide a securement device that has the capability of remaining with the catheter during the catheter's life without becoming dislodged. A securement device that is present during complete dwell period of a catheter can ensure continuous securement of the catheter. Also, it can ensure catheter securement during site cleaning (migration and dislodgement of the catheter is a concern during a dressing change where normally a securement device is also removed and the catheter has no securement).

### SUMMARY OF THE INVENTION

It should be appreciated that this Summary is provided to introduce a selection of concepts in a simplified form, the concepts being further described below in the Detailed Description. This Summary is not intended to identify key features or essential features of this disclosure, nor is it intended to limit the scope of the invention.

According to some embodiments of the present invention, a catheter securement device includes a first arm having opposite proximal and distal ends, a second arm having opposite proximal and distal ends, and a coupler secured to the proximal end of each of the first and second arms. A first prong extends laterally from the first arm distal end, and a first gripping handle extends laterally from the first arm between the first arm proximal and distal ends. A second prong extends laterally from the second arm distal end, and a second gripping handle extends laterally from the second arm between the second arm proximal and distal ends.

The coupler is configured to maintain the first and second arms in generally parallel, spaced apart relationship when the first and second arms are in in a relaxed state. The coupler includes an internal bore configured to receive a catheter tube therethrough. The coupler may also be configured to receive and support a catheter hub.

When the first and second gripping handles are pushed toward each other by a user (e.g., a healthcare provider/technician, caretaker, etc.), the first and second arms are moved to an unrelaxed state such that the first and second prongs overlap each other to facilitate insertion and removal of the first and second prongs through an incision in the skin of a patient. Upon insertion through the incision in the skin of the patient and subsequent release of the gripping handles by the user, the first and second arms return to their relaxed state such that the first and second prongs become subcutaneously anchored in the skin of the patient.

In some embodiments, the first prong extends laterally in a first direction and the second prong extends laterally in a second direction that is opposite to the first direction. In some embodiments, the first prong and the second prong are substantially co-planar when the first and second arms are in their relaxed state. In some embodiments, the first gripping handle and the second gripping handle are substantially co-planar when the first and second arms are in their relaxed state. In some embodiments, the first prong, the second prong, the first gripping handle and the second gripping handle are substantially co-planar when the first and second arms are in their relaxed state.

According to some embodiments of the present invention, a catheter securement device includes a tubular member having a distal end, a sleeve secured (e.g., hingedly secured) to the tubular member distal end, and a pair of resilient arms that extend outward from a distal end of the sleeve. The tubular member and sleeve are configured to receive a catheter tube therethrough.

Each arm includes a prong extending laterally from a distal end thereof. A pair of buttons extend through opposite sides of the sleeve and each button is connected to a respective arm. When the pair of buttons are depressed by a user (e.g., a healthcare provider/technician, caretaker, etc.), the pair of arms are moved toward each other to an unrelaxed state such that the prongs overlap each other to facilitate insertion and removal of the prongs through an incision in the skin of a patient. Upon insertion through the incision in the skin of the patient and subsequent release of the buttons by the user, the arms return to their relaxed state such that the prongs become subcutaneously anchored in the skin of the patient.

In some embodiments, the prong of one of the arms extends laterally in a first direction and the prong of the other one of the arms extends laterally in a second direction that is opposite to the first direction. In some embodiments, the prongs of the pair of arms are substantially co-planar when the pair of arms are in their relaxed state.

According to some embodiments of the present invention, a catheter securement device includes a flexible base that defines a compartment for receiving a catheter hub, and a cover pivotably secured to the base and that is movable between an open position exposing the compartment and a closed position covering the compartment. The base includes first and second portions, each having opposite first and second surfaces and opposite first and second ends. The first and second portions are substantially co-planar when the base is in a relaxed state. The first and second portions are foldable along a centerline of the base to an unrelaxed state by a user, e.g., a healthcare provider/technician, caretaker, etc. The second surface of the first portion and the second surface of the second portion are in face-to-face relationship when the first and second portions are folded to the unrelaxed state;

A first arm extends outward from the first end of the first portion and includes a prong that extends laterally from a distal end of the first arm. The second arm extends outward from the first end of the second portion and includes a prong that extends laterally from a distal end of the second arm. When the first and second portions are in the relaxed state, the prong of the first arm extends laterally in a first direction and the prong of the second arm extends laterally in a second direction that is opposite from the first direction. When the first and second portions are folded by a user to the unrelaxed state, the prong of the first arm and the prong of the second arm are in adjacent relationship and each extends in substantially a same direction to facilitate insertion and removal of the prong of the first arm and the prong of the second arm through an incision in the skin of a patient. Upon insertion through the incision in the skin of the patient and subsequently allowing the first and second portions to return to the relaxed state, the prongs of the first and second arms become subcutaneously anchored in the skin of the patient.

In some embodiments, the prong of the first arm and the prong of the second arm are substantially co-planar when the first and second portions are in their relaxed state.

In some embodiments, the first portion of the base includes a first elongate slot and a first guide post that is slidably engaged therewithin, and the second portion of the base includes a second elongate slot and a second guide post that is slidably engaged therewithin. The first and second posts extend outwardly from the respective first and second portions and are each configured to engage a respective aperture in a wing of a catheter hub placed within the compartment and such that the catheter hub is adjustably movable within the compartment.

In some embodiments, one of the first or second slots includes teeth configured to engage and lock in place the respective post slidably engaged therewith.

According to some embodiments of the present invention, a catheter securement device includes a base having a first section, a second section, and an elastomeric member positioned between and removably secured to the first and second sections. The first section has an inner edge, an opposite outer edge, a front edge, an opposite rear edge, a first surface, and an opposite second surface. Similarly, the second section has an inner edge, an opposite outer edge, a front edge, an opposite rear edge, a first surface, and an opposite second surface. The elastomeric member is removably secured to the first section inner edge and to the second section inner edge. In some embodiments, the elastomeric member includes a first slot formed in a first side thereof that is configured to slidably retain the inner edge of the first section therein, and a second slot formed in an opposite second side thereof that is configured to slidably retain the inner edge of the second section therein. The first and second sections are substantially co-planar when the elastomeric member is in a relaxed state. The elastomeric member is foldable along a centerline thereof to an unrelaxed state such that the second surface of the first section and the second surface of the second section are in face-to-face relationship.

A first flexible hinge member is secured to a portion of the first section front edge and includes a first arm extending outward from a front end thereof. The first arm includes a prong extending laterally from a distal end thereof. A second flexible hinge member is secured to a portion of the second section front edge and includes a second arm extending outward from a front end thereof. The second arm includes a prong extending laterally from a distal end thereof. When the elastomeric member is in the relaxed state, the prong of the first arm extends laterally in a first direction and the prong of the second arm extends laterally in a second direction that is different (e.g., opposite, etc.) from the first direction. When the elastomeric member is folded to the unrelaxed state by a user, e.g., a healthcare provider/technician, caretaker, etc., the prong of the first arm and the prong of the second arm are in adjacent relationship and each extends in substantially a same direction to facilitate insertion and removal of the first and second prongs through an incision in the skin of a patient. Upon insertion through the incision in the skin of the patient and subsequently allowing the elastomeric member to return to the relaxed state, the prongs of the first and second arms become subcutaneously anchored in the skin of the patient. The first and second flexible hinge members are configured to allow the base to pivot upwardly away from the skin of a patient to allow for cleaning of the catheter insertion site while maintaining the prongs of the first and second arms subcutaneously anchored in the skin of the patient.

In some embodiments, the prong of the first arm and the prong of the second arm are substantially co-planar when the elastomeric member is in its relaxed state.

The catheter securement device also includes a housing that is removably secured to the base. The housing includes opposite front and rear walls, opposite side walls, and a floor that define a compartment. The front wall includes a first opening, and the rear wall includes a second opening, and the first and second openings are generally aligned with one another. An elastomeric element is removably positioned within the compartment and includes a passageway that extends from the first opening in the housing front wall to the second opening in the housing rear wall. The passageway is configured to receive and secure a catheter tube therein. When the elastomeric element is removed from the compartment, the compartment is configured to receive a catheter hub therewithin.

In some embodiments, the first section includes a first guide rail on the first surface thereof and the second section includes a second guide rail on the first surface thereof. A bottom surface of the housing includes a first feature configured to slidingly engage the first guide rail and a second feature configured to slidingly engage the second guide rail such that the housing is movable relative to the base. The catheter securement device also includes a cover that is pivotably secured to the housing and is movable between an open position exposing the compartment and a closed position covering the compartment. Movement of the housing relative to the base is restricted when the cover is in the closed position.

According to some embodiments of the present invention, a catheter securement device includes a base having a first section and second section that are removably secured to each other. The first section has an inner edge, an opposite outer edge, a front edge, an opposite rear edge, a first surface, and an opposite second surface. Similarly, the second section has an inner edge, an opposite outer edge, a front edge, an opposite rear edge, a first surface, and an opposite second surface. The first section inner edge abuts the second section inner edge and the first section and second section are maintained in a substantially co-planar configuration by a locking mechanism that removably engages the first section and the second section.

A first flexible hinge member is secured to a portion of the first section front edge. A first arm extends outward from a front end of the first hinge member. The first arm includes a first resilient prong at a distal end thereof. A second flexible hinge member is secured to a portion of the second section front edge. A second arm extends outward from a front end of the second hinge member. The second arm includes a second resilient prong at a distal end thereof. Prior to attachment of the device to a patient, an enclosure is removably secured to the first and second flexible hinge members and includes an elongate tube that is configured to retain the first arm and first prong in an unrelaxed configuration (e.g., a generally straight configuration), and to retain the second arm and second prong in an unrelaxed configuration (e.g., a generally straight configuration) to facilitate insertion of the first and second prongs through an incision in the skin of a patient. When the enclosure is removed by a user, e.g., a healthcare provider/technician, caretaker, etc., the first prong is configured to return to a relaxed state extending laterally from the first arm in a first direction, and the second prong is configured to return to a relaxed state extending laterally from the second arm in a second direction different from the first direction. Upon insertion through the incision in the skin of the patient and subsequent removal of the enclosure, the first and second prongs become subcutaneously anchored in the skin of the patient. The first and second flexible hinge members are configured to allow the base to pivot upwardly away from the skin to allow for cleaning at the catheter insertion site while maintaining the first and second prongs subcutaneously anchored in the skin of the patient.

In some embodiments, the first prong and second prong are substantially co-planar when in their respective relaxed state.

The catheter securement device also includes a housing that is removably secured to the base. The housing includes opposite front and rear walls, opposite side walls, and a floor that define a compartment. The front wall includes a first opening, and the rear wall includes a second opening. The first and second openings are generally aligned with one another. An elastomeric element is removably positioned within the compartment and includes a passageway extending from the first opening in the housing front wall to the second opening in the housing rear wall. The passageway is configured to receive and secure a catheter tube therein. When the elastomeric element is removed from the compartment, the compartment is configured to receive a catheter hub therewithin.

In some embodiments, the first section includes a first guide rail on the first surface thereof and the second section includes a second guide rail on the first surface thereof. A bottom surface of the housing includes a first feature configured to slidingly engage the first guide rail and a second feature configured to slidingly engage the second guide rail such that the housing is movable relative to the base. The catheter securement device also includes a cover that is pivotably secured to the housing and is movable between an open position exposing the compartment and a closed position covering the compartment. Movement of the housing relative to the base is restricted when the cover is in the closed position.

According to some embodiments of the present invention, a catheter securement device includes a base having opposite front and rear edges, opposite side edges, a first surface, and an opposite second surface. A first elastomeric element is secured to the base first surface and includes a plurality of spaced apart elastomeric walls extending outwardly therefrom. The plurality of spaced apart walls define a passageway configured to receive and secure a catheter tube therein. In addition, the plurality of spaced apart walls are configured to deform and support a catheter hub thereon.

An elongate member is slidably secured to the base and is movable between a retracted position where a distal end of the elongate member is adjacent the base front edge and an extended position where the distal end of the elongate member is extended outwardly from the base front edge. A first flexible hinge member is secured to the distal end of the elongate member, and a second flexible hinge member is secured to the distal end of the elongate member adjacent the first flexible hinge. The first flexible hinge member includes a first arm extending outward from a front end thereof. The first arm includes a first resilient prong at a distal end thereof. The second flexible hinge member includes a second arm extending outward from a front end thereof. The second arm includes a second resilient prong at a distal end thereof. In some embodiments, the first arm and first prong, and the second arm and second prong, are substantially co-planar when in their respective relaxed states.

An enclosure is removably secured to the first and second flexible hinge members and includes an elongate tube that is configured to retain the first arm and first prong in an unrelaxed configuration (e.g., a generally straight configuration), and to retain the second arm and second prong in an unrelaxed configuration (e.g., a generally straight configuration) to facilitate insertion of the first and second prongs through an incision in the skin of a patient. When the enclosure is removed by a user, e.g., a healthcare provider/technician, caretaker, etc., the first prong is configured to return to a relaxed state extending laterally from the first arm in a first direction, and the second prong is configured to return to a relaxed state extending laterally from the second arm in a second direction different from the first direction. Upon insertion through the incision in the skin of the patient and subsequent removal of the enclosure, the first and second prongs become subcutaneously anchored in the skin of the patient. The first and second flexible hinge members are configured to allow the base to pivot upwardly away from the skin to allow for cleaning at the catheter insertion site while maintaining the first and second prongs subcutaneously anchored in the skin of the patient.

A cover is removably secured to the base and includes openings to facilitate passage therethrough of a catheter tube secured to the first elastomeric element and/or a catheter hub positioned on the first elastomeric element.

According to some embodiments of the present invention, a catheter securement device includes a base having a first section and a second section movably coupled together. The first and second sections are movable between a first configuration wherein the first and second sections are substantially co-planar and a second configuration wherein the first and second sections are in face-to-face relationship. A first wall extends outwardly from a first surface of the first section and a second wall extends outwardly from a first surface of the second section. The first and second walls are in adjacent spaced apart relationship and are configured to receive and secure a catheter tube therein when the first and second sections are in the first configuration. A cover is removably secured to the base and includes openings to facilitate passage therethrough of a catheter tube secured between the first and second walls.

The first and second sections are movably secured together by a hinge assembly that includes a first hinge member that rotates in response to movement of the second section, and a second hinge member that rotates in response to movement of the first section. A first arm extends outward from a front end of the first hinge member and includes a first prong at a distal end thereof. A second arm extends outward from a front end of the second hinge member and includes a second prong at a distal end thereof. When the first and second sections are moved to the second configuration, the prong of the first arm and the prong of the second arm are in adjacent relationship and each extends in substantially the same direction to facilitate insertion and removal of the prong of the first arm and the prong of the second arm through an incision in the skin of a patient. Upon insertion through the incision in the skin of the patient and subsequently moving the first and second sections to the first configuration by a user, the prongs of the first and second arms become subcutaneously anchored in the skin of the patient.

In some embodiments, the first arm has a first length and the second arm has a second length that is different from the first length.

In some embodiments, the second hinge member includes a longitudinal bore and the first hinge member is a cylindrical pin positioned within the longitudinal bore and freely rotatable relative to each other.

In some embodiments, the second hinge member has a semicylindrical configuration with a longitudinal channel. The first hinge member is a cylindrical pin that is rotatably positioned within the longitudinal channel of the second hinge member. The first and second hinge members are freely rotatable relative to each other. A retaining member surrounds the first and second hinge members and is configured to prevent the pin of the first hinge member from dislodging from the longitudinal channel of the second hinge member during use of the catheter securement device. In some embodiments, the retaining member is elastomeric and has a tubular shape with a longitudinal slit formed therethrough on one side and a handle extending outward from an opposite side. The retaining member is configured to be removed from the catheter securement device to facilitate separation of the first and second hinge members by a user gripping the handle and pulling the retaining member such that first and second hinge members pass through the slit.

According to some embodiments of the present invention, a catheter securement device includes a base having a first section and a second section, and an elastomeric member removably attached to one of the first or second sections. The elastomeric member includes a channel or passageway formed therein that is configured to receive a catheter tube. The passageway may have various shapes. For example, in some embodiments, the passageway may be generally straight. In other embodiments, the passageway may have an arcuate shape. The elastomeric member also includes at least one recessed portion formed therein adjacent to the passageway.

The device includes a hinge assembly that has a first hinge member attached to the first section of the base, and a second hinge member attached to the second section of the base. The first hinge member and the second hinge member are operably connected such that the first and second sections are movable between a first configuration where the first and second sections and the elastomeric member are substantially co-planar and a second configuration where the first and second sections are in face-to-face relationship.

A first arm extends outward from a front end of the first hinge member and includes a first prong at a distal end thereof. A second arm extends outward from a front end of the second hinge member and includes second prong at a distal end thereof. In some embodiments, the first arm has a first length and the second arm has a second length that is different from the first length.

When the first and second sections are moved to the second configuration, the prong of the first arm and the prong of the second arm are in adjacent relationship and each extends in substantially the same direction to facilitate insertion and removal of the prong of the first arm and the prong of the second arm through an incision in the skin of a patient. Upon insertion through the incision in the skin of the patient and subsequently moving the first and second sections to the first configuration, the prongs of the first and second arms become subcutaneously anchored in the skin of the patient.

A cover is configured to be secured to the base and includes openings to facilitate passage therethrough of a catheter tube secured within the passageway. The cover includes at least one protrusion that is configured to engage the at least one recessed portion in the elastomeric member. Engagement of the at least one protrusion with the at least one recessed portion squeezes the passageway and facilitates retention of the catheter in the passageway and prevents unwanted movement of the catheter. In some embodiments, the elastomeric member includes a first recessed portion formed therein on one side of the passageway, and a second recessed portion formed therein on an opposite side of the passageway. The cover includes a first protrusion that is configured to engage the first recessed portion and a second protrusion that is configured to engage the second recessed portion when the cover is secured to the base. The engagement of the first protrusion with the first recessed portion and the engagement of the second protrusion with the second recessed portion squeezes the passageway and facilitates retention of the catheter in the passageway and prevents unwanted movement of the catheter.

It is noted that aspects of the invention described with respect to one embodiment may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail below.

### BRIEF DESCRIPTION OF THE DRAWINGS

The accompanying drawings, which form a part of the specification, illustrate various embodiments of the present invention. The drawings and description together serve to fully explain embodiments of the present invention.

FIGS. 1A-1D are schematic illustrations of a device for subcutaneously anchoring a catheter securement device to a patient, according to some embodiments of the present invention.

FIGS. 2A-2B are schematic illustrations of a device for subcutaneously anchoring a catheter securement device to a patient, according to some embodiments of the present invention.

FIG. 3 is a perspective view of a catheter securement device configured to be subcutaneously secured to a patient, according to some embodiments of the present invention.

FIGS. 4A-4B are perspective views of a catheter securement device, according to some embodiments of the present invention.

FIG. 5 is a perspective view of a catheter securement device configured to be subcutaneously anchored to a patient, according to some embodiments of the present invention.

FIG. 6 is a schematic illustration of a technique for removing a subcutaneously anchored catheter securement device from a patient, according to some embodiments of the present invention.

FIG. 7 illustrates a technique for subcutaneously anchoring a catheter to a patient, according to some embodiments of the present invention.

FIGS. 8A-8C are perspective views of a catheter securement device configured to be subcutaneously anchored to a patient, according to some embodiments of the present invention.

FIGS. 9-10 are perspective views of clips configured to be subcutaneously anchored to a patient and that capture and release the wings of a catheter hub, according to some embodiments of the present invention.

FIGS. 11-12 illustrate a clip configuration for retaining a catheter hub, according to some embodiments of the present invention.

FIG. 13A is a plan view of a catheter securement device configured to be subcutaneously anchored to a patient, according to some embodiments of the present invention.

FIG. 13B is a perspective view of the catheter securement device of FIG. 13A.

FIG. 13C is a perspective view of a coupler configured to support the first and second arms of the catheter securement device of FIG. 13A.

FIG. 14 is a perspective view of the catheter securement device of FIG. 13A supporting a catheter hub.

FIG. 15 is a perspective view of a catheter securement device configured to be subcutaneously anchored to a patient, according to some embodiments of the present invention.

FIG. 16 illustrates the catheter securement device of FIG. 15 supporting a catheter hub.

FIG. 17 is a perspective view of a catheter securement device configured to be subcutaneously anchored to a patient, according to some embodiments of the present invention.

FIG. 18 is a cross-sectional view of the catheter securement device of FIG. 17 taken along line 18-18.

FIG. 19 is an enlarged partial view of the proximal end portion of the catheter securement device of FIG. 17.

FIG. 20 is an enlarged partial view of the distal end portion of the catheter securement device of FIG. 17.

FIGS. 21-24 are perspective views of a catheter securement device, according to some embodiments of the present invention.

FIG. 25 is an end view of the catheter securement device of FIG. 23 taken along line 25-25.

FIG. 26 is a perspective view of the catheter securement device of FIGS. 21-24 illustrating movement of the catheter hub therewithin.

FIG. 27 is a perspective view of a catheter securement device configured to be subcutaneously anchored to a patient, according to some embodiments of the present invention.

FIG. 28 is a perspective view of a section of the base of the catheter securement device of FIG. 27.

FIG. 29 is an exploded perspective view of the base of the catheter securement device of FIG. 27.

FIG. 30 is a perspective view of the base of the catheter securement device of FIG. 27.

FIG. 31 is a perspective view of the catheter securement device of FIG. 27 with the housing cover open and illustrating the housing compartment.

FIG. 32 is a partial end view of the catheter securement device of FIG. 31 taken along line 32-32.

FIG. 33 is a perspective view of the catheter securement device of FIG. 27 with the housing cover closed.

FIG. 34 is a partial perspective view of the catheter securement device of FIG. 27 illustrating the cover latch mechanism.

FIG. 35 is a perspective view of the catheter securement device of FIG. 27 with a catheter tube secured within the housing compartment.

FIG. 36 is a perspective view of the catheter securement device of FIG. 27 with a catheter tube hub secured within the housing compartment.

FIG. 37 is a side view of the catheter securement device of FIG. 27 illustrating movement of the base and housing about the flexible hinge members.

FIG. 38 is a perspective view of a catheter securement device configured to be subcutaneously anchored to a patient, according to some embodiments of the present invention.

FIG. 39 is a perspective view of the base of the catheter securement device of FIG. 38.

FIG. 40 is a partial enlarged perspective view of a portion of the catheter securement device of FIG. 38 illustrating, in phantom line, an enclosure that surrounds the first and second arms.

FIGS. 41A-41B are perspective views of the base of the catheter securement device of FIG. 38 illustrating movement of the locking mechanism securing the first and second sections together.

FIG. 42 is an exploded perspective view of the base of the catheter securement device of FIG. 38 illustrating separation of the first and second sections.

FIG. 43 is a perspective view of a catheter securement device configured to be subcutaneously anchored to a patient, according to some embodiments of the present invention. An enclosure that surrounds the first and second arms is illustrated in phantom line.

FIG. 44 is a perspective view of the catheter securement device of FIG. 43 with the enclosure surrounding the first and second arms removed.

FIG. 45 is a perspective view of the catheter securement device of FIG. 43 with the member, to which the first and second arms are secured, moved to an extended position.

FIG. 46 illustrates the catheter securement device of FIG. 43 with a catheter tube secured between the flexible walls of the base.

FIG. 47 illustrates the catheter securement device of FIG. 46 with the cover secured thereto.

FIG. 48 illustrates the catheter securement device of FIG. 43 with a catheter tube hub placed on top of the flexible walls of the base.

FIG. 49 illustrates the catheter securement device of FIG. 48 with the cover secured thereto.

FIG. 50 is a perspective view of a catheter securement device configured to be subcutaneously anchored to a patient, according to some embodiments of the present invention.

FIG. 51 illustrates the catheter securement device of FIG. 50 with a catheter tube secured between the walls of the base.

FIG. 52 illustrates the catheter securement device of FIG. 51 with the cover secured thereto.

FIGS. 53A-53C are perspective views of the catheter securement device of FIG. 50 illustrating movement of the first and second sections of the base.

FIG. 54 is a partial perspective view of a catheter securement device configured to be subcutaneously anchored to a patient, according to some embodiments of the present invention.

FIG. 55 is a perspective view of the catheter securement device of FIG. 54 illustrating a retaining member surrounding the first and second hinge members, according to some embodiments of the present invention.

FIG. 56 is a perspective view of the catheter securement device of FIG. 55 with the first and second sections in to face-to-face relationship so that the prongs of the arms are in a configuration for insertion into a patient.

FIG. 57 illustrates the catheter securement device of FIG. 55 with a catheter tube secured between the walls of the base.

FIG. 58 illustrates the catheter securement device of FIG. 57 with the cover secured thereto.

FIG. 59 is an enlarged partial perspective view of the retaining member of FIG. 55 surrounding the first and second hinge members.

FIG. 60 is an exploded perspective view of the catheter securement device of FIG. 55 with the retaining member removed therefrom.

FIG. 61 is an exploded perspective view of a catheter securement device configured to be subcutaneously anchored to a patient, according to some embodiments of the present invention.

FIG. 62A illustrates the catheter securement device of FIG. 61 with the first and second sections of the base in substantially co-planar relationship.

FIG. 62B illustrates the catheter securement device of FIG. 62A with the first and second sections of the base in face-to-face relationship so that the prongs of the arms are in a configuration for insertion into a patient.

FIG. 63 illustrates the catheter securement device of FIG. 61 with a catheter tube secured to the base and a cover secured to the base.

FIG. 64 is a perspective view of the inside surface of the cover of FIG. 63.

FIG. 65 is a perspective view of a pair of first and second arms that can be utilized with the various catheter securement devices of the present invention.

### DETAILED DESCRIPTION OF THE INVENTION

The present invention will now be described more fully hereinafter with reference to the accompanying figures, in which embodiments of the present invention are shown. The present invention may, however, be embodied in many different forms and should not be construed as limited to the embodiments set forth herein.

Embodiments of the present invention are directed to catheter securement solutions that help minimize catheter migration and dislodgement during the dressing change of a catheter, while also not elevating other clinical burden, for example, microbial growth, patient pain, and significant workflow impact. Embodiments of the present invention provide catheter securement devices that are configured to hold a catheter or catheter hub near the insertion site of the catheter in a patient's skin. The devices allow for 360° site cleaning without increasing the risk of catheter movement during dressing change of the catheter, such as long-dwelling IV catheters, for example, a peripherally inserted central catheter (PICC) line and a central venous catheter (CVC) line.

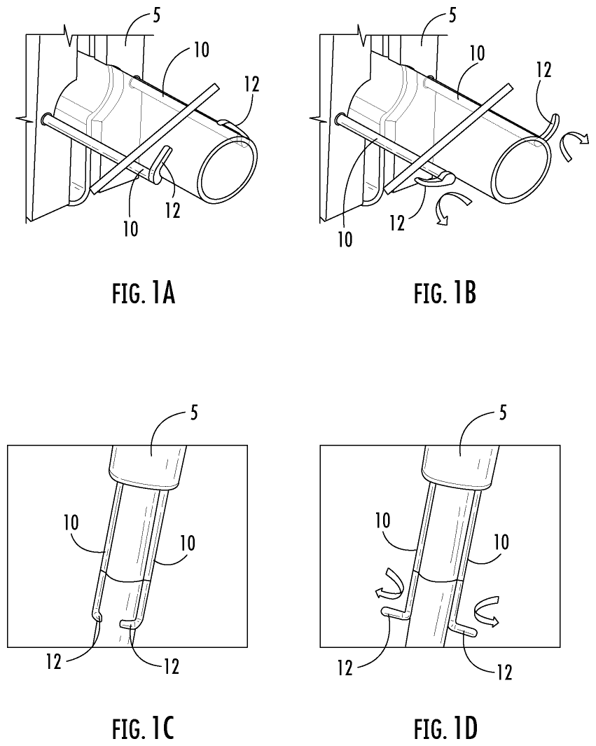

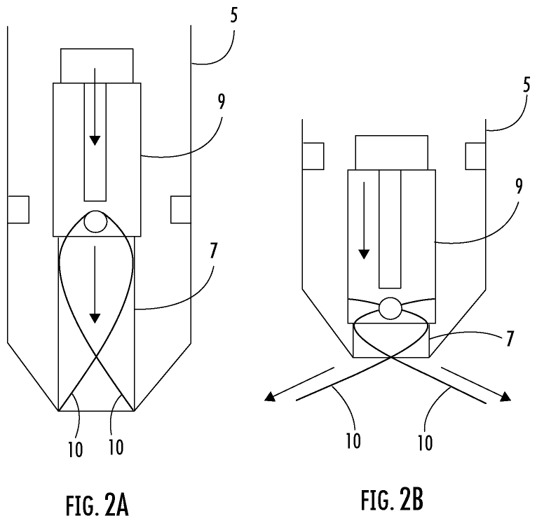

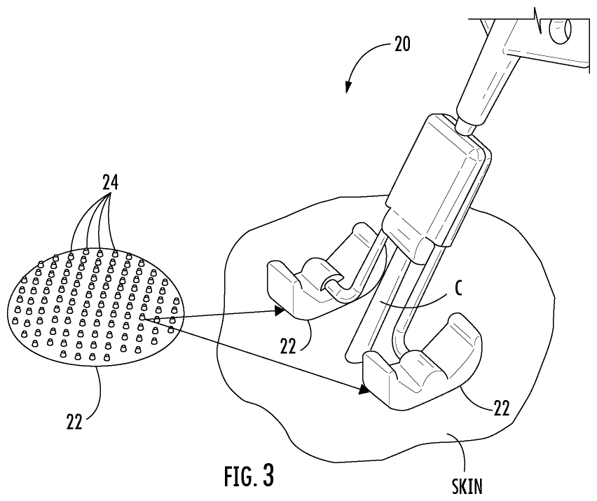

FIGS. 1A-1D, 2A-2B and 3 show different techniques for subcutaneously anchoring a catheter securement device 5. FIGS. 1A-1D illustrate a technique in which arms 10 attached to the securement device 5 are inserted into a patient, then rotated such that fingers or prongs 12 at the end of each arm 10 spread out to maintain the device in place. FIGS. 2A-2B schematically illustrate a catheter securement device 5 in which a hollow or solid needle 7 is inserted into the patient (FIG. 2A), and movement of a plunger 9 within the needle 7 forces spring-loaded semi-rigid arms 10 out of the end of the needle 7, where they deflect radially outwardly (FIG. 2B) to maintain the device 5 in place. FIG. 3 shows a device 20 with pads 22 located on both sides of the catheter C. The pads 22 have microneedles 24 (see inset of FIG. 3) that penetrate the patient's skin and hold the device 20 in place. Other techniques will also be apparent to those of skill in this art (e.g., using sutures rather than microneedles).

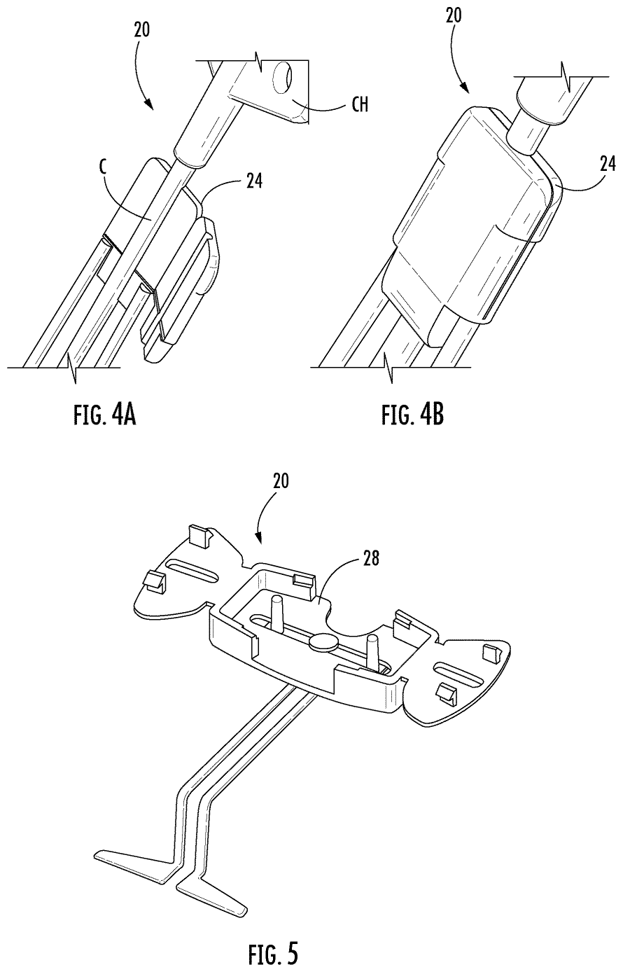

Once the securement device 20 is anchored in place subcutaneously, the device 20 is then mounted to the catheter C. This can be achieved, for example, by mounting the device 20 to the catheter C itself, which is exemplified in FIGS. 4A-4B by including a clip 24 on the securement device that receives a portion of the catheter C available between the insertion site and the catheter hub CH, or by mounting the device 20 to the hub CH of the catheter C. In FIG. 5, the device 20 includes a compartment 28 with features that mate with and retain a hub CH therein. In each instance, the securement device 20 can be easily removed (e.g., unclipped) from the catheter C or hub CH when necessary.

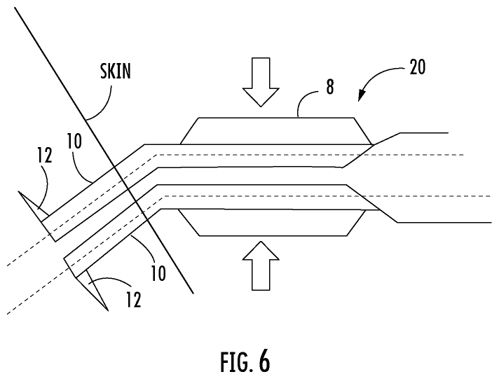

FIG. 6 illustrates a catheter securement device 20 wherein the prongs 12 at the end of movable arms 10 are subcutaneously anchored into the skin of a patient.

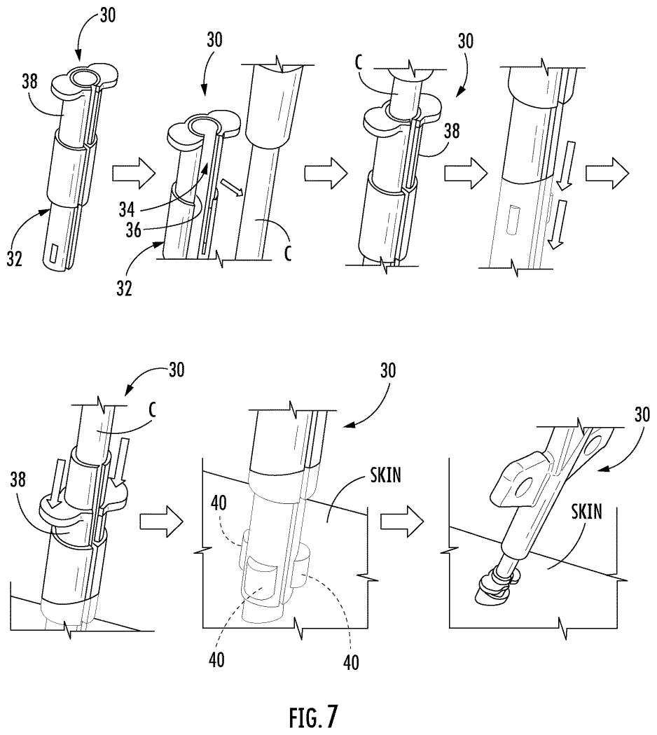

FIG. 7 illustrates a technique for securing a catheter securement device 30 via one or more inflatable balloons 40. The device 30 has an elongate body 32 with a hollow central cavity 34 accessible by a longitudinal slot 36. Inflatable balloons 40 are located at the lower end of the device 30. A piston 38 is received in the body 32 and extends upwardly therefrom. As shown in FIG. 7, the device 30 is slipped onto the shaft of a catheter C so that the catheter C is located in the central cavity 34. The catheter C and device 30 are then inserted into the patient. The piston 38 is then lowered relative to the body 32, which causes the balloons 40 to inflate (or, as an alternative, prongs to extend/rotate) and project radially outwardly from the body 32 under the patient's skin. The inflated balloons 40 act as anchors to maintain the device 30 in place.

The device 30 can be removed by raising the piston 38 relative to the body 32, which causes the balloons 40 to deflate to their original configuration. The device 30 and catheter C can then be removed from the patient.

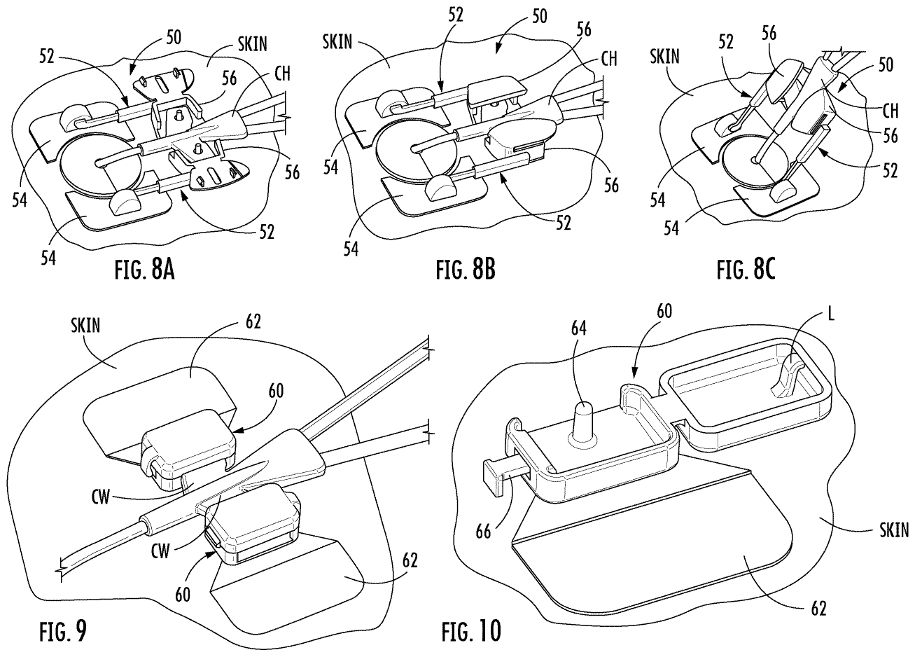

FIGS. 8A-8C illustrate a securement device 50 that includes two telescoping arms 52 that each pivotally attach to a respective pad 54 on either side of the insertion site. Clips 56 are present at the opposite ends of the arms 52. The clips 56 are configured to receive and capture the wings of a catheter hub CH. The telescoping nature of the arms 52 enables the catheter C and hub CH to be lifted relative to the pads 54 for cleaning around the insertion site (shown in FIG. 8C).

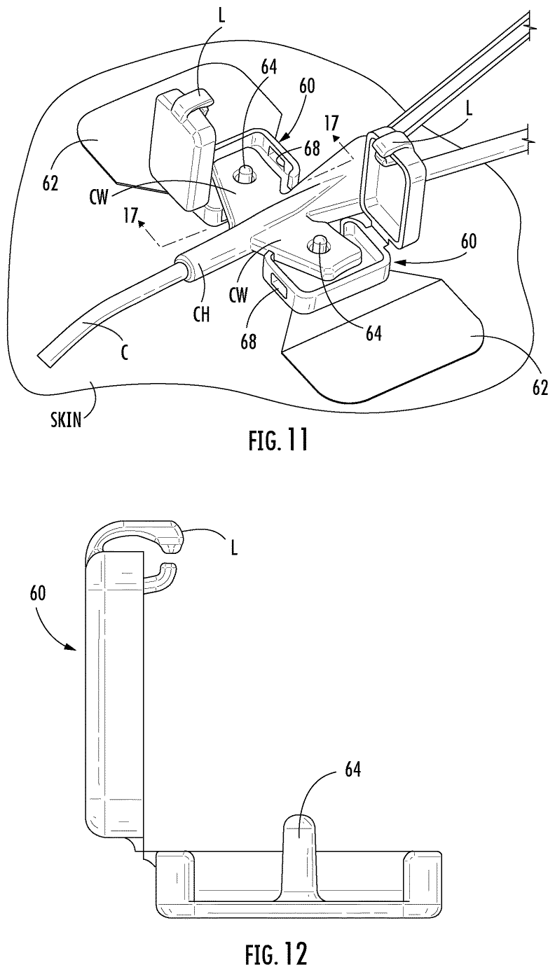

FIGS. 9 and 10 illustrate clips 60 that pivot to capture and release the wings CW of a catheter hub CH. Each of the clips 60 is attached to a pad 62 that is attached to the patient's skin. The lower half of the clip (FIG. 10) includes a post 64 that can be received in a hole in the wing CW, and also includes a sliding member 66 on one end. The upper half of the clip includes a latch L that can be captured by the lower half near the sliding member 66 when the clip 60 is closed. The sliding member 66 can be used to deflect the latch L out of the way in order for the closed clip 60 to be allowed to open. FIGS. 11 and 12 illustrate another clip configuration in which a latch L on the upper half of the clip 60 latches to a slot 68 in the end wall of the lower half of the clip 60.

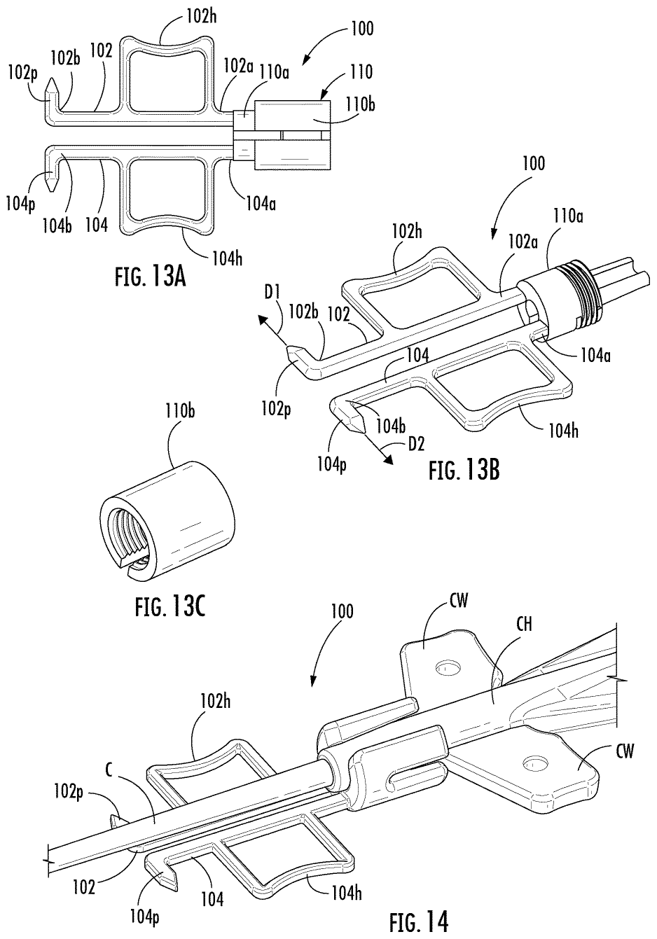

Referring to FIGS. 13A-13C and 14, a catheter securement device 100, according to some embodiments of the present invention, includes a first arm 102 having opposite proximal and distal ends 102a, 102b, a second arm 104 having opposite proximal and distal ends 104a, 104b, and a coupler 110 secured to the proximal end 102a, 104a of each of the first and second arms 102, 104. A first prong 102p extends laterally from the first arm distal end 102b, and a first gripping handle 102h extends laterally from the first arm 102 between the first arm proximal end 102a and distal end 102b. Similarly, a second prong 104p extends laterally from the second arm distal end 104b, and a second gripping handle 104h extends laterally from the second arm 104 between the second arm proximal end 104a and distal end 104b.

The coupler 110 is configured to maintain the first and second arms 102, 104 in generally parallel, spaced apart relationship when the first and second arms 102, 104 are in in a relaxed state, as illustrated in FIGS. 13A and 13B. The coupler 110 includes an internal bore 112 configured to receive a catheter tube C therethrough. The coupler 110 may also be configured to receive and support a catheter hub CH. In the illustrated embodiment, the coupler 110 includes a first externally threaded portion 110a that is secured to the proximal end 102a, 104a of the arms 102, 104, and a second internally threaded portion 110b that threadingly engages the first portion 110a, as shown in FIG. 13A.

The first and second arms 102, 104 are formed from material having resiliency in that, when each arm is moved from the relaxed state (FIG. 13A) to an unrelaxed state, the arm wants to return to its relaxed state. When the first and second gripping handles 102h, 104h are pushed toward each other by a user (e.g., a healthcare provider/technician, caretaker, etc.), the first and second arms 102, 104 are moved to an unrelaxed state such that the first and second prongs overlap each other. This overlap facilitates insertion and removal of the first and second prongs 02p, 104p through an incision in the skin of a patient. Upon insertion through the incision in the skin of the patient and subsequent release of the gripping handles 102h, 104h by the user, the first and second arms 102, 104 return to the relaxed state such that the first and second prongs 102p, 104p become subcutaneously anchored in the skin of the patient.

In some embodiments, the gripping handles 102h, 104h may be formed from, or covered with, an elastomeric material to facilitate gripping by a user.

In the illustrated embodiment, the first prong 102p extends laterally in a first direction D1 and the second prong 104p extends laterally in a second direction D2 that is different (e.g., opposite, etc.) to the first direction D1. In the illustrated embodiment, the first prong, 102p the second prong 104p, the first gripping handle 102h and the second gripping handle 104 are substantially co-planar when the first and second arms are in the relaxed state. However, in other embodiments, the first prong 102p and the second prong 104p may have other orientations relative to each other as well as to the gripping handles 102h, 104h. Similarly, in other embodiments, the first gripping handle 102h and the second gripping handle 104h may have other orientations relative to each other.

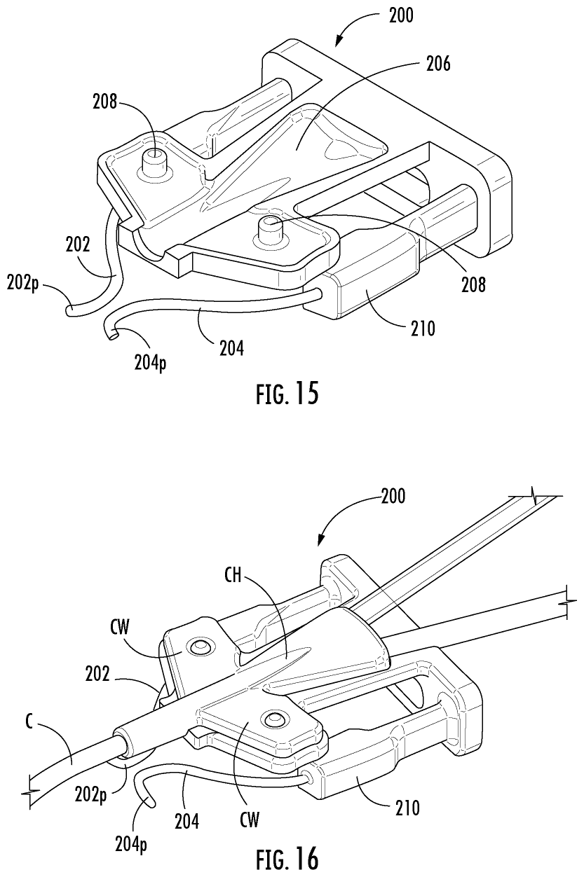

FIGS. 15 and 16 illustrate another catheter securement device 200 having movable arms 202, 204 and respective prongs 202p, 204p configured to be inserted under the patient's skin. The prongs 202p, 204p are mounted on the ends of flexible arms 202, 204 that are cantilevered from a base 210. A hub mounting panel 206 also extends from the base 210; it has two small posts 208 for capturing the holes in the wings CW of a catheter hub CH (FIG. 16). The flexible arms 202, 204 can be pressed toward each other during insertion and removal of the prongs 202p, 204p in the patient.

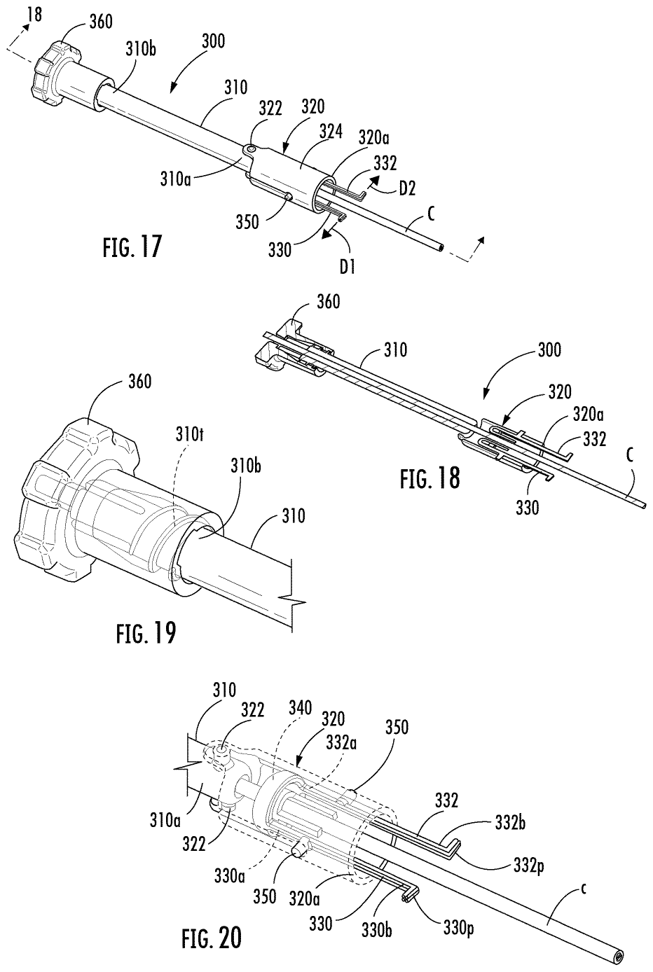

Referring to FIGS. 17-20, a catheter securement device 300 according to some embodiments of the present invention is illustrated. The illustrated catheter securement device 300 includes a tubular member 310 having a distal end 310a and a sleeve 320 secured to the tubular member distal end 310a. The sleeve 320 has a generally cylindrical shape, although other shapes are possible. The sleeve 320 has an inner diameter that is greater than an outer diameter of the distal end 310a of the tubular member 310, the sleeve 320 being received over the distal end 310a such that the sleeve 320 and the tubular member 310 are generally concentric. The sleeve 320 is hingedly or pivotably secured to the tubular member 310 by a pair of pivot members 322 that extend through aligned openings formed in the end portion 310a of the tubular member 310 and in a wall 324 of the cylindrical sleeve 320, the pivot members 322 defining a pivot axis substantially perpendicular to a longitudinal axis of the tubular member 310, as would be understood by one of skill in the art. However, other ways of movably securing the sleeve 320 to the tubular member 310 may be utilized. The clearance between the inner surface of the sleeve 320 and the outer surface of the end portion 310a of the tubular member 310 permits relative angular displacement of the sleeve 320 about the pivot axis while maintaining the sleeve 320 and tubular member 310 in a generally concentric relationship such that a catheter tube C can extend therethrough, as illustrated.

A pair of resilient arms extend 330, 332 outward from a distal end 320a of the sleeve 320. Each arm 330, 332 includes a respective prong 330p, 332p that extends laterally from a distal end 330b, 332b thereof. The proximal ends 330a, 332a of the arms 330, 332 are fixedly secured to a member 340 within the sleeve 320, as illustrated in FIG. 20. A pair of buttons 350 extend through opposite sides of the wall 324 of the sleeve 320, as illustrated, and each button 350 is connected to a respective arm 330, 332. When the pair of buttons 350 are depressed by a user (e.g., a healthcare provider/technician, caretaker, etc.), the pair of arms 330, 332 are moved toward each other to an unrelaxed state such that the prongs 330p, 332p overlap each other to facilitate insertion and removal of the prongs 330p, 332p through an incision in the skin of a patient. Upon insertion through the incision in the skin of the patient and subsequent release of the buttons 350 by the user, the resilient arms 330, 332 return to a relaxed state such that the prongs 330p, 332p become subcutaneously anchored in the skin of the patient.

In the illustrated embodiment, the prong 330p of the first arm 330 extends laterally in a first direction D1 and the prong 332p of the second arm 332 extends laterally in a second direction D2 that is opposite to the first direction D1. However, in other embodiments, the prong 330p of the first arm 330 and the prong 332p of the second arm 332 may have other orientations relative to each other. Also, in the illustrated embodiment, the prong 330p of the first arm and the prong 332p of the second arm 332 are substantially co-planar when the first and second arms 330, 332 are in the relaxed state. However, in other embodiments, the prong 330p of the first arm and the prong 332p of the second arm 332 may be oriented in different planes.

In the illustrated embodiment, the proximal end 310b of the tubular member 310 has threads 310t and a series of extensions. A nut 360 with a tapered inner surface can be threaded onto the tubular member 310; this action causes deflection of the extensions into engagement with the catheter C to mount the device onto the catheter C.

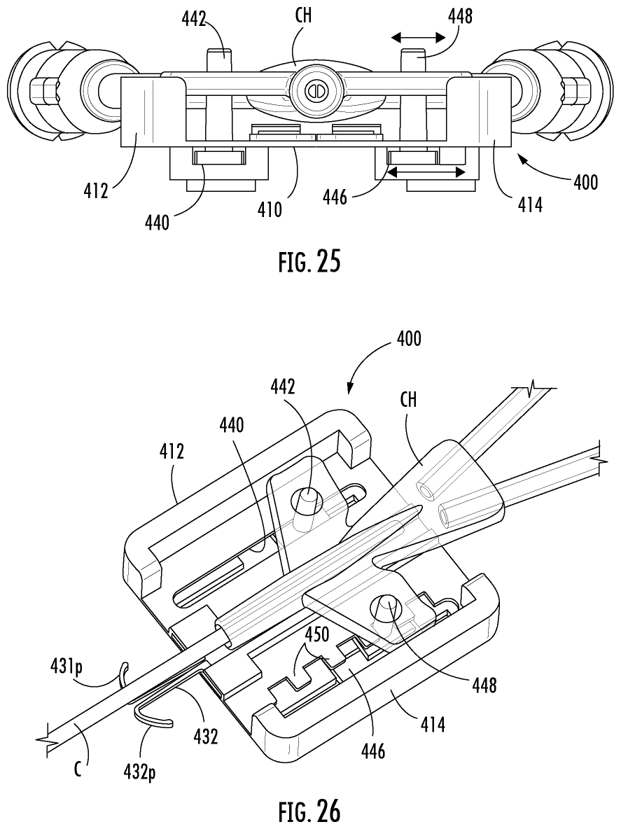

Referring to FIGS. 21-26, a catheter securement device 400 according to some embodiments of the present invention is illustrated. The illustrated catheter securement device 400 includes a flexible base 410 that defines a compartment 420 for receiving a catheter hub CH, and a cover 460 pivotably secured to the base 410. The cover 460 is movable between an open position (FIG. 21) exposing the compartment 420 and a closed position covering the compartment 420, typically after a catheter hub CH has been secured within the compartment 420. The base 410 includes first and second portions 412, 414. The first portion 412 includes opposite first and second surfaces 412a, 412b and opposite first and second ends 412c, 412d, and the second portion 414 includes opposite first and second surfaces 414a, 414b and opposite first and second ends 414c, 414d.

The first and second portions 412, 414 of the flexible base 410 are substantially co-planar when the base 410 is in a relaxed state. The first and second portions 412, 414 are foldable along a centerline CL of the base 410 to an unrelaxed state by a user, e.g., a healthcare provider/technician, caretaker, etc. For example, to facilitate insertion of the arms 430, 432, described below, into a patient near a catheter insertion site, the first and second portions 412, 414 are folded along the centerline CL so that the flexible base 410 is in an unrelaxed state and with the second surface 412b of the first portion 412 and the second surface 414b of the second portion 414 in face-to-face relationship (not shown).

A first arm 430 extends outward from the first end 412c of the first portion 412 and includes a prong 430p that extends laterally from a distal end 430b of the first arm 430. The second arm 432 extends outward from the first end 414c of the second portion 414 adjacent to the first arm 430 and includes a prong 432p that extends laterally from a distal end 432b of the second arm 432. When the base 410 and first and second portions 412, 414 are in the relaxed state, as illustrated in FIGS. 21-26, the prong 430p of the first arm 430 extends laterally in a first direction D1 and the prong 432p of the second arm 432 extends laterally in a second direction D2 that is different from (e.g., opposite from, etc.) the first direction D1. Also, in the illustrated embodiment, the prong 430p of the first arm and the prong 432p of the second arm 432 are substantially co-planar when the base 410 is in the relaxed state. However, in other embodiments, the prong 430p of the first arm and the prong 432p of the second arm 432 may be oriented in different planes.

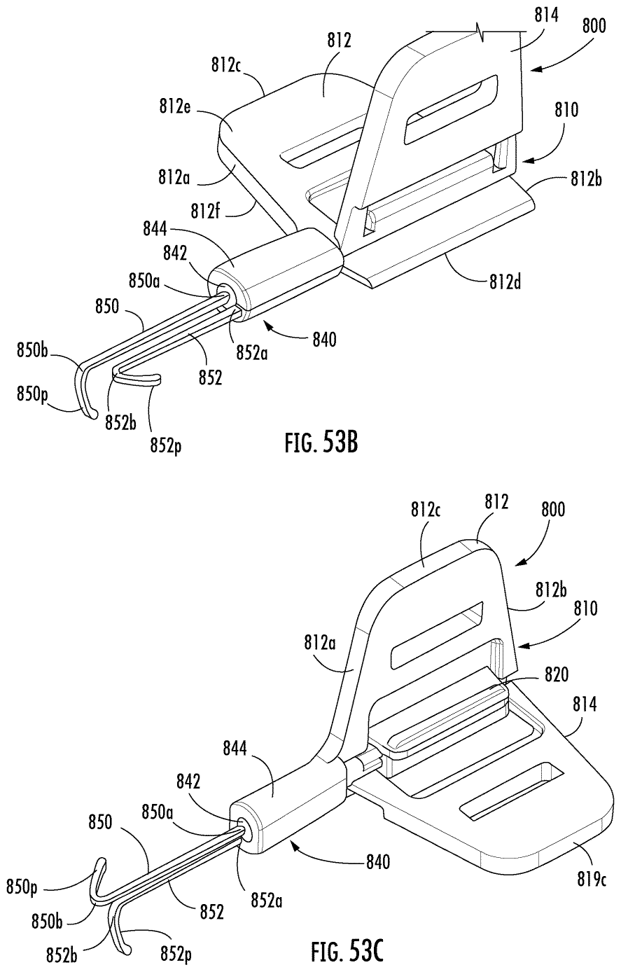

When the first and second portions 412, 414 are folded by a user along the centerline CL such that they are in face-to-face relationship, the prong 430p of the first arm 430 and the prong 432p of the second arm 432 are in adjacent relationship and each extends in substantially a same direction to facilitate insertion and removal of the prong 430p of the first arm 430 and the prong 432p of the second arm 432 through an incision in the skin of a patient. Although not illustrated, the orientation of the arms 430, 432 and prongs 430p, 432p would be similar to that illustrated in FIG. 53A, except the arms 430, 432 would be in adjacent relationship and not overlapping relationship. Upon insertion through the incision in the skin of the patient and subsequently allowing the first and second portions to return to the relaxed state, the prongs 430p, 432p of the first and second arms 430, 432 become subcutaneously anchored in the skin of the patient.

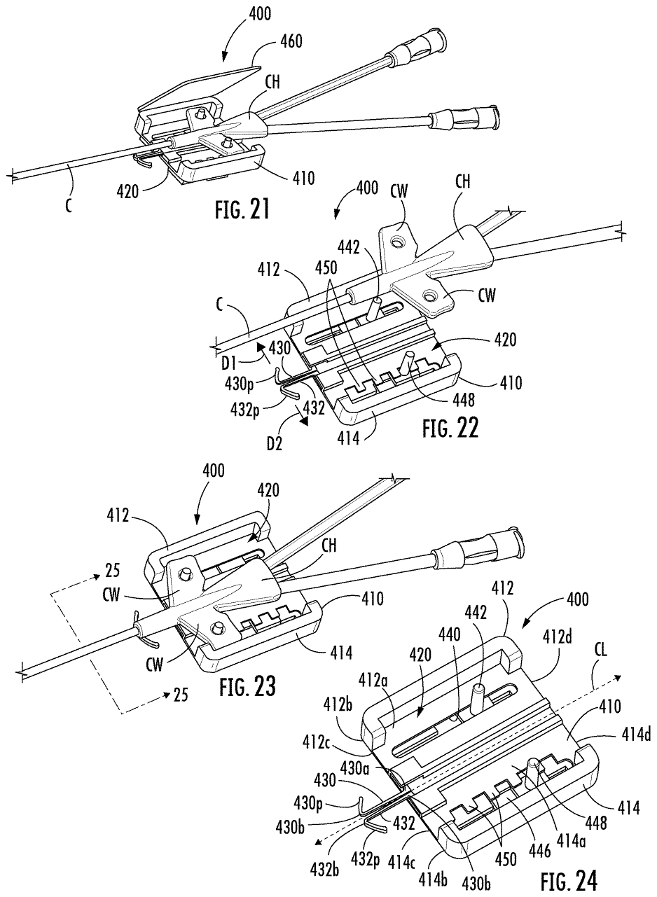

In the illustrated embodiment, the first portion 412 of the base 410 includes a first elongate slot 440 and a first guide post 442 slidably engaged therewithin, and the second portion 414 of the base 410 includes a second elongate slot 446 and a second guide post 448 slidably engaged therewithin. The first and second guide posts 442, 448 extend outwardly from the respective first and second portions 412, 414 and are each configured to engage respective apertures in a wing CW of a catheter hub CH placed within the compartment 420 (FIG. 23) and such that the catheter hub CH is adjustably movable within the compartment. The second slot 446 includes a plurality of spaced apart teeth 450 configured to engage and lock in place the second guide post 448 that is slidably engaged therewith. The distance between the first and second slots 440, 446 is such that, when a catheter hub CH is mounted onto the guide posts 442, 448, the post 448 in the “toothed” slot 446 is drawn inwardly (FIG. 25) so that the post 448 is locked into place. The cover 460 of the catheter securement device 400 is then pivoted to cover the catheter hub CH.

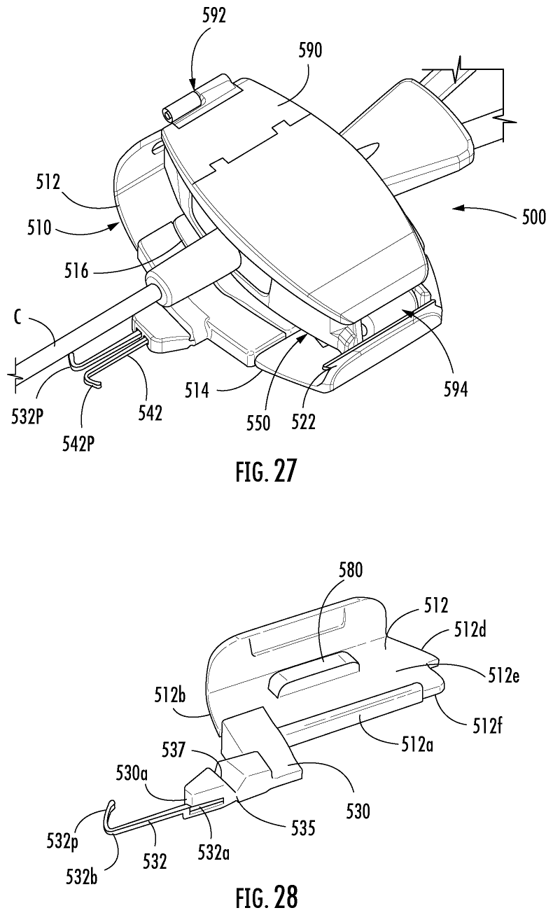

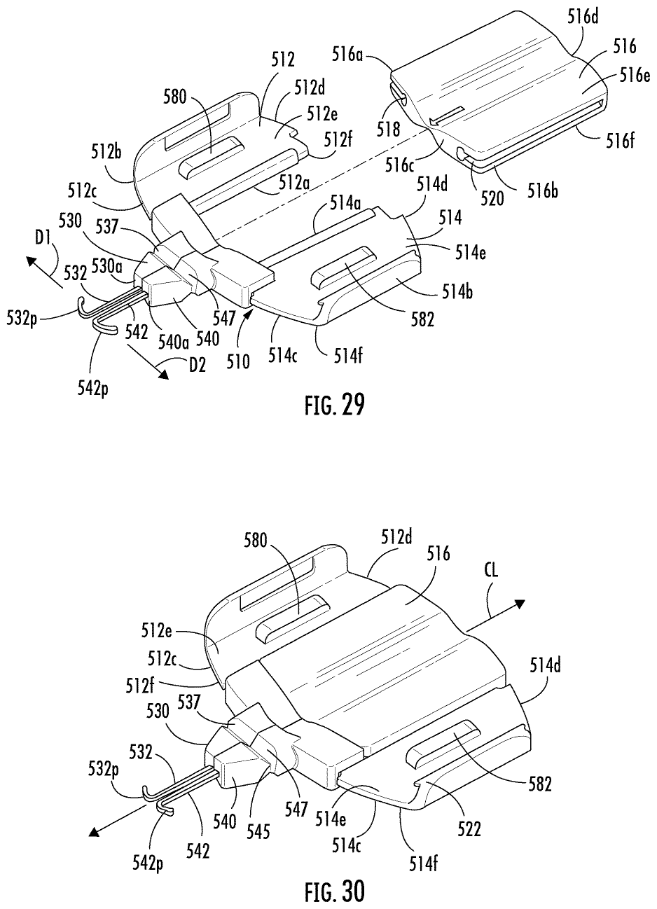

Referring to FIGS. 27-37, a catheter securement device 500 according to some embodiments of the present invention is illustrated. The illustrated catheter securement device 500 includes a base 510 having a first section 512, a second section 514, and an elastomeric member 516 positioned between and removably secured to the first and second sections 512, 514. The first section 512 has an inner edge 512a, an opposite outer edge 512b, a front edge 512c, an opposite rear edge 512d, a first surface 512e, and an opposite second surface 512f. Similarly, the second section 514 has an inner edge 514a, an opposite outer edge 514b, a front edge 514c, an opposite rear edge 514d, a first surface 514e, and an opposite second surface 514f. The elastomeric member 516 is removably secured to the first section inner edge 512a and to the second section inner edge 514a, as illustrated.

The elastomeric member 516 includes opposite first and second sides 516a, 516b, opposite first and second ends 516c, 516d, and opposite first and second surfaces 516e, 516f. A first slot 518 is formed in the first side 516a of the elastomeric member 516 and a second slot 520 is formed in the second side 516b of the elastomeric member 516. The first slot 518 is shaped and configured to slidably retain the inner edge 512a of the first section 512 therein, and the second slot 520 is shaped and configured to slidably retain the inner edge 514a of the second section therein. The first and second sections 512, 514 are substantially co-planar when the elastomeric member 516 is in a relaxed state. The elastomeric member 516 is foldable along a centerline CL thereof. To facilitate insertion of the arms 532, 542, described below, into a patient near a catheter insertion site, the elastomeric member 516 is folded along the centerline CL to an unrelaxed state such that the second surface 512f of the first section 512 and the second surface 514f of the second section 514 are in face-to-face relationship (not shown).

The illustrated catheter securement device 500 includes a first flexible hinge member 530 secured to a portion of the first section front edge 512c and a second flexible hinge member 540 secured to a portion of the second section front edge 514c. A first arm 532 extends outward from a front end 530a of the first hinge member 530. The first arm 532 includes a prong 532p that extends laterally from a distal end 532b of the arm 532. An opposite proximal end 532a of the first arm is secured within a portion of the first flexible hinge member 530, as illustrated. A second arm 542 extends outward from a front end 540a of the second hinge member 540. The second arm 542 includes a prong 542p that extends laterally from a distal end 542b of the second arm 542. An opposite proximal end 542a of the second arm 542 is secured within a portion of the second flexible hinge member 540.

When the elastomeric member 516 is in the relaxed state, as illustrated in FIGS. 29, 30, 32), the prong 532p of the first arm 532 extends laterally in a first direction D1 and the prong 542p of the second arm 542 extends laterally in a second direction D2 that is different from (e.g., opposite from, etc.) the first direction D1. Also, in the illustrated embodiment, the prong 532p of the first arm 530 and the prong 542p of the second arm 542 are substantially co-planar when the elastomeric member 516 is in the relaxed state, as illustrated in FIG. 32. However, in other embodiments, the prong 532p of the first arm 530 and the prong 542p of the second arm 542 may be oriented in different planes and may extend in other directions relative to each other.

When the elastomeric member 516 is folded along the centerline CL by a user, e.g., a healthcare provider/technician, caretaker, etc., such that the second surface 512f of the first section 512 and the second surface 514f of the second section 514 are in face-to-face relationship, the prong 532p of the first arm 532 and the prong 542p of the second arm 542 are in adjacent relationship and each extends in substantially a same direction to facilitate insertion and removal of the first and second prongs 532p, 542p through an incision in the skin of a patient. Although not illustrated, the orientation of the arms 530, 532 and prongs 530p, 532p would be similar to that illustrated in FIG. 53A, except the arms 530, 532 would be in adjacent relationship and not overlapping relationship. Upon insertion through the incision in the skin of the patient, a user subsequently allows the elastomeric member 516 to return to its relaxed state and the prongs 532p, 542p of the first and second arms 532, 542 become subcutaneously anchored in the skin of the patient.

The first and second flexible hinge members 530, 540 are configured to allow the base 510 and housing 550 (described below), that supports a catheter tube or catheter hub therein, to pivot upwardly away from the skin of a patient while maintaining the prongs 532p, 542p of the first and second arms 532, 542 subcutaneously anchored in the skin of the patient (FIG. 37). This allows for cleaning around the insertion site of a catheter supported by the device 500. In the illustrated embodiment, a medial portion 535 of the first flexible hinge member 530 has a V-shaped notch 537 that defines a flexure region and, similarly, a medial portion 545 of the second flexible hinge member 540 has a V-shaped notch 547 that defines a flexure region. Each notch 537, 547 lowers the bending stiffness of the respective hinge member 530, 540 such that the hinge members 530, 540 bend easily at their respective medial portions 535, 545.

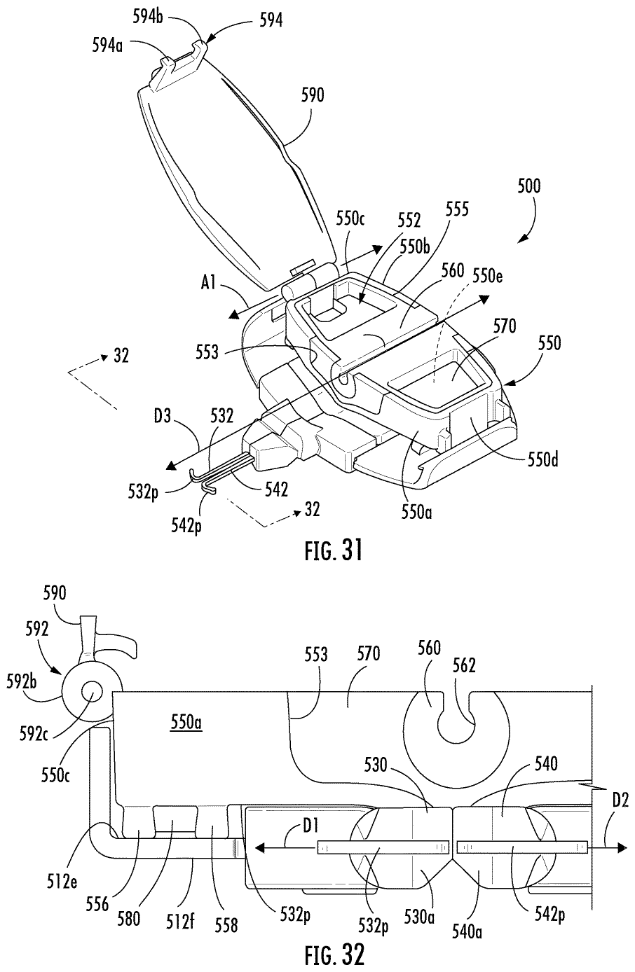

The illustrated catheter securement device 500 also includes a housing 550 that is removably secured to the base 510 when the elastomeric member 516 is in its relaxed state and the first and second sections 512, 514 are substantially co-planar, as illustrated in FIGS. 27 and 31. The illustrated housing 550 includes opposite front and rear walls 550a, 550b, opposite side walls 550c, 550d, and a floor 550e that define a compartment 552. The compartment 552 may have various configurations and shapes and is not limited to the illustrated configuration. For example, the illustrated front and rear walls 550a, 550b are curved in shape. However, in other embodiments, the front and rear walls 550a, 550b may be straight in shape, or even concave in shape.

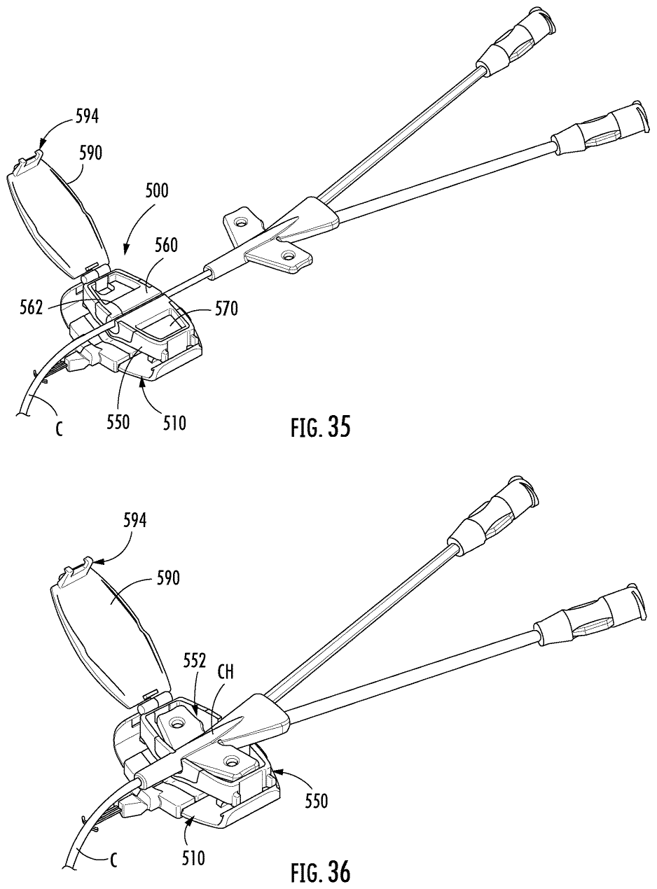

The front wall 550a includes a first opening 553, and the rear wall 550b includes a second opening 555 that are generally aligned with one another along illustrated direction D3. A first elastomeric element 560 is removably positioned within the compartment 552 and includes a passageway 562 that extends from the first opening 553 in the housing front wall 550a to the second opening 555 in the housing rear wall 550b. The passageway 562 is configured to receive and secure a catheter tube C therein, as illustrated in FIG. 35. A second elastomeric element 570 is positioned within the compartment 552 beneath the first elastomeric element 560, as illustrated. The second elastomeric element 570 is configured to support a catheter hub CH when the first elastomeric element 560 is removed from the compartment 552, as illustrated in FIG. 36.

The housing 550 is movably secured to the base 510 so as to be adjustably positioned on the base (i.e., the housing is movable along direction D3). In the illustrated embodiment, the first section 512 of the base 510 includes a first guide rail 580 on its first surface 512e, and the second section 514 of the base 510 includes a second guide rail 582 on its first surface 514e. A first pair of spaced apart walls 556, 558 extend downwardly from the bottom surface 551 of the housing 550 adjacent the housing side wall 550c, and a second pair of spaced apart walls 556, 558 extend downwardly from the bottom surface 551 of the housing 550 adjacent the housing side wall 550d, as illustrated. Each pair of walls 556, 558 is configured to slidably receive a respective guide rail 580, 582 therebetween, as illustrated in FIG. 32, and allow the housing 550 to be movable relative to the base 510 along the guide rails 580, 582.

In the illustrated embodiment, each guide rail 580, 582 has opposed outer side walls that taper outwardly along at least a portion of its height. Each of the walls 556, 558 has correspondingly tapered inner walls so that, during insertion, a respective rail 580, 582 is cammed between the spaced apart walls 556, 558 until a snap-fit condition is reached, as illustrated in FIG. 32. This snap-fit condition allows the housing 550 to be removably secured to the base 510 and allows the housing 550 to be moved relative to the base 510 along the guide rails 580, 582.

A cover 590 is pivotably secured to the housing 550 via a hinge assembly 592. In the illustrated embodiment, the hinge assembly 592 includes a first hinge portion 592a secured to the cover 590, a second hinge portion 592b secured to the housing side wall 550c, and a pivot member 592c, such as a pin, received by the cooperating hinge features of the first and second hinge portions 592a, 592b. The pivot member 592c provides pivotal coupling of the cover 590 to the housing 550 about the hinge axis A1 such that the cover 590 is movable between open and closed positions.

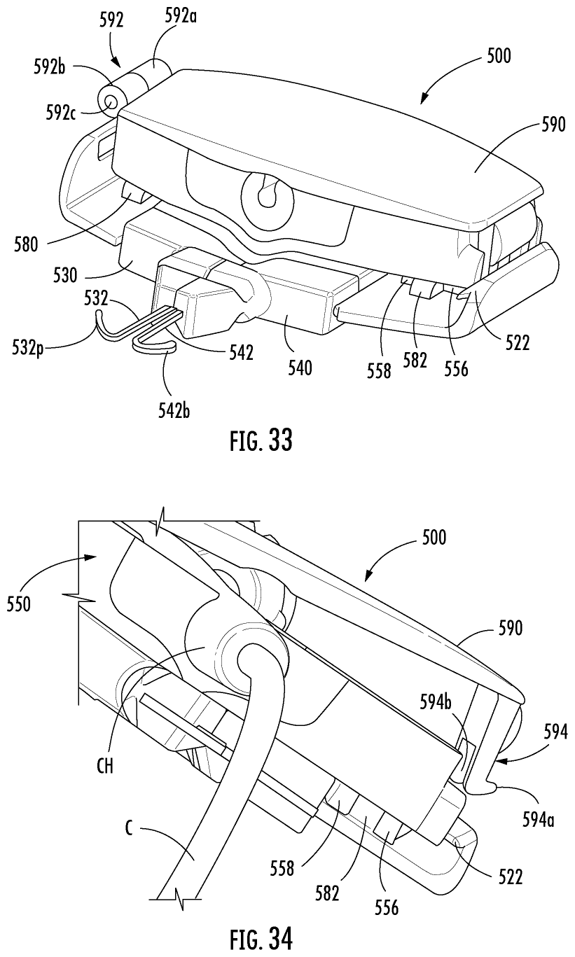

The illustrated cover 590 also includes a latch mechanism 594 that is configured to releasably secure the cover 590 to the housing 550 when in the closed position (FIG. 33).

The illustrated latch mechanism 594 includes a pair of latch members 594a, 594b connected to the cover 590. The latch members 594a, 594b are configured to move over a retaining feature 522 at the outer edge 514b of the base 510 as the cover 590 is brought toward a closed position. The latch members 594a, 594b deflect as they pass over the retaining feature 522 and then resiliently engage behind the retaining feature 522 to releasably secure the cover 590 to the housing 550. When the cover 590 is in the closed position, the cover 590 provides a downward force on the housing 550 that is sufficient to restrict movement of the housing 550 relative to the base 510. When the cover 590 is moved to an open position, the housing 550 can be moved relative to the base 510.

FIG. 36 illustrates a catheter hub CH supported within the compartment 552 of the housing (i.e., with the first elastomeric element 560 removed from the compartment 552) and with the cover 590 in an open position such that the housing 550 and catheter hub CH can be moved relative to the base 510. FIG. 27 illustrates the cover 590 in the closed position such that the housing 550 and catheter hub CH cannot be moved relative to the base 510.

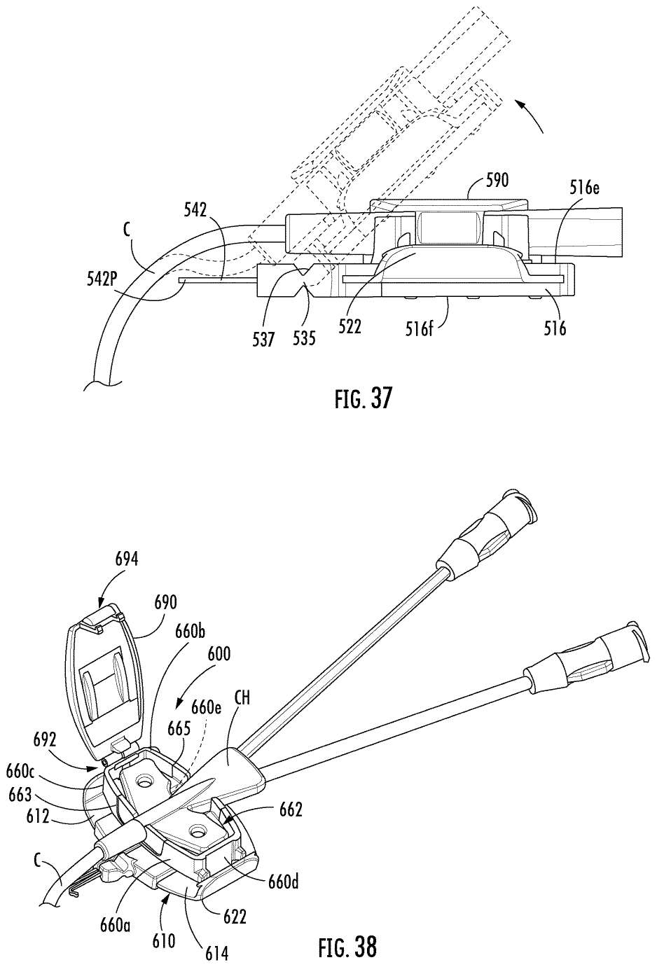

Referring to FIGS. 38-42, a catheter securement device 600 according to some embodiments of the present invention is illustrated. The illustrated catheter securement device 600 includes a base 610 having a first section 612 and a second section 614 that are removably secured to each other. The first section 612 has an inner edge 612a, an opposite outer edge 612b, a front edge 612c, an opposite rear edge 612d, a first surface 612e, and an opposite second surface 612f. Similarly, the second section 614 has an inner edge 614a, an opposite outer edge 614b, a front edge 614c, an opposite rear edge 614d, a first surface 614e, and an opposite second surface 614f. When assembled as the base 610, the first section inner edge 612a abuts the second section inner edge 614a and the first section 612 and the second section 614 are maintained in a substantially co-planar configuration by a locking mechanism 616 that removably engages the first section 612 and the second section 614.

In the illustrated embodiment, the first section 612 includes a locking rail 618a on the first surface 612e adjacent to the inner edge 612a, and the second section 614 includes a locking rail 618b on the first surface 614e adjacent to the inner edge 614a. The locking rail 618a on the first surface 612e of the first section is longer than the locking rail 618b on the first surface 614e of the second section 614. The locking mechanism 616 is slidably secured to the locking rail 618a on the first section 612 via a respective first slot 616a formed in the bottom surface of the locking mechanism 616. The locking mechanism 616 is prevented from dislodging from the locking rail 618a by the enlarged end portion 619 of the locking rail 618a.

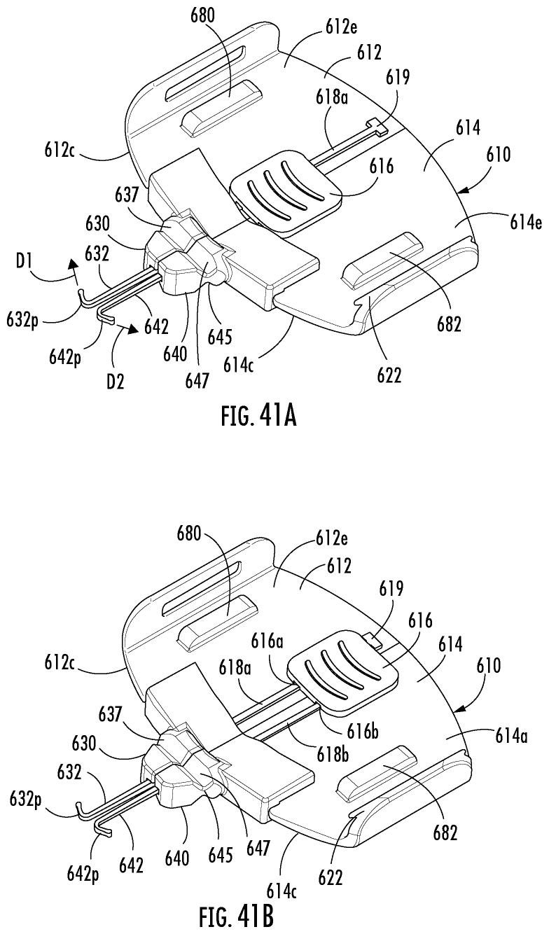

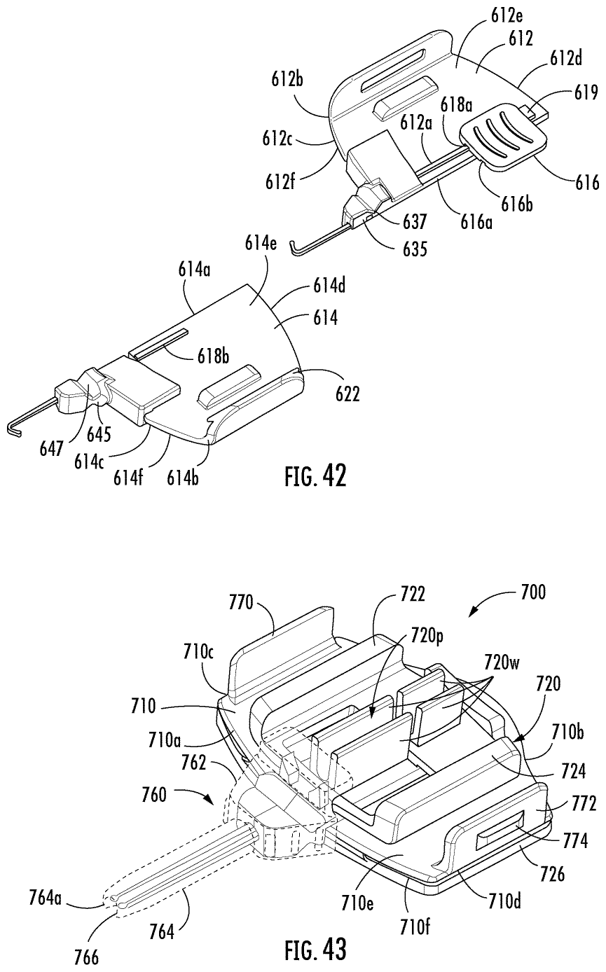

The locking mechanism 616 includes a second slot 616b formed in the bottom surface thereof in adjacent relationship with the first slot 616a. The second slot 616b is configured to engage the locking rail 618b on the second section 614 and to retain the first and second sections 612, 614 in substantially co-planar relationship. FIG. 41B illustrates the first and second sections 612, 614 aligned with the inner edges 612a, 614a of each section in abutting relationship and with the locking mechanism 616 beginning engagement with the locking rail 618b of the second section 614. In FIG. 41A, the locking mechanism 616 is fully engaged with the locking rail 618b of the second section 614.

The illustrated catheter securement device 600 includes a first flexible hinge member 630 secured to a portion of the first section front edge 612c and a second flexible hinge member 640 secured to a portion of the second section front edge 614c. A first arm 632 extends outward from a front end 630a of the first hinge member 630. The first arm 632 includes a resilient prong 632p that extends laterally from a distal end 632b of the arm 632. An opposite proximal end 632a of the first arm is secured within a portion of the first flexible hinge member 630, as illustrated. A second arm 642 extends outward from a front end 640a of the second hinge member 640. The second arm 642 includes a resilient prong 642p that extends laterally from a distal end 642b of the second arm 642. An opposite proximal end 642a of the second arm 642 is secured within a portion of the second flexible hinge member 640.

The first and second flexible hinge members 630, 640 are configured to allow the base 610 and housing 660 (described below), that supports a catheter tube or catheter hub therein, to pivot upwardly away from the skin of a patient while maintaining the prongs 632p, 642p of the first and second arms 632, 642 subcutaneously anchored in the skin of the patient, in a similar manner as illustrated above with respect to the embodiment in FIG. 37. This allows for cleaning around the insertion site of a catheter supported by the device 600. In the illustrated embodiment, a medial portion 635 of the first flexible hinge member 630 has a V-shaped notch 637 that defines a flexure region and, similarly, a medial portion 645 of the second flexible hinge member 640 has a V-shaped notch 647 that defines a flexure region. Each notch 637, 647 lowers the bending stiffness of the respective hinge member 630, 640 such that the hinge members 630, 640 bend easily at their respective medial portions 635, 645.

FIGS. 41A-41B and 42 illustrate the first and second arms 632, 642 and their respective resilient prongs 632p, 642p in a relaxed state. When in the relaxed state, the prong 632p of the first arm 532 extends laterally in a first direction D1 and the prong 642p of the second arm 642 extends laterally in a second direction D2 that is different from the first direction D1. Also, in the illustrated embodiment, the prong 632p of the first arm 630 and the prong 642p of the second arm 642 are substantially co-planar when in the relaxed state. However, in other embodiments, the prong 632p of the first arm 630 and the prong 642p of the second arm 642 may be oriented in different planes.

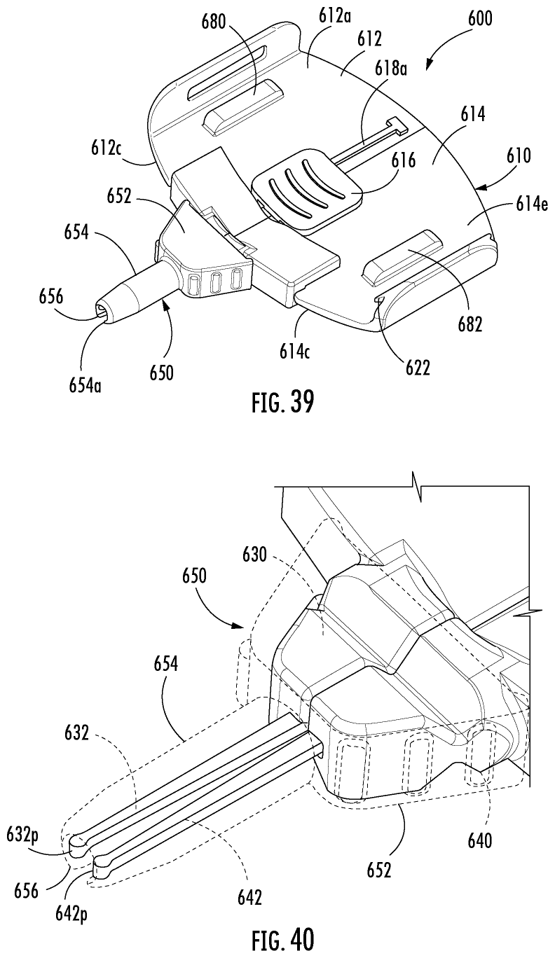

Referring to FIGS. 39 and 40, the catheter securement device 600 includes an enclosure 650 that is removably secured to the first and second flexible hinge members 630, 640 prior to attachment of the catheter securement device 600 to a patient. The illustrated enclosure 650 includes a first portion 652 that snugly fits over the first and second flexible hinge members 630, 640 from above, and a second portion 654 extending outwardly from the first portion 652. In the illustrated embodiment, the second portion 654 is an elongate tube with an open distal end 654a and a slot 656 formed therein that extends from the open distal end 654a along the entire length of the tube, as illustrated. The second portion 654 is configured to retain the first arm 632 and first prong 632p in an unrelaxed, generally straight configuration, and to retain the second arm 642 and second prong 642p in an unrelaxed, generally straight configuration to facilitate insertion of the first and second prongs 632p, 642p through an incision in the skin of a patient. The second portion 654 has an atraumatic profile so that it can pass through an incision in a patient with minimal risk of snagging, lacerating, or otherwise damaging patient tissue.

When attaching the catheter securement device 600 to a patient, the second portion 654 is inserted into an incision in a patient, and the enclosure 650 is then removed from the device 600 by a user, such as a healthcare provider/technician, caretaker, etc. The user lifts up on the enclosure 650 to remove the first portion 652 from the first and second flexible hinge members 630, 640 and the second portions 654 from surrounding the first and second arms and respective prongs 632p, 642p. This causes the first prong 632p of the first arm 632 to return to a relaxed state extending laterally from the first arm 632 in a first direction, and the second prong 642p of the second arm 642 to return to a relaxed state extending laterally from the second arm 642 in a second direction different from the first direction. As a result, the first and second prongs 632p, 642p become subcutaneously anchored in the skin of the patient. The first and second flexible hinge members 630, 640 are configured to allow the base to pivot upwardly away from the skin while maintaining the first and second prongs subcutaneously anchored in the skin of the patient, as described above.

Similar to the catheter securement device 500, described above, the catheter securement device 600 also includes a housing 660 that is removably secured to the base 610. The housing 660 includes opposite front and rear walls 660a, 660b, opposite side walls 660c, 660d, and a floor 660e that define a compartment 662. The front wall 660a includes a first opening 663, and the rear wall 660b includes a second opening 665. The first and second openings 663, 665 are generally aligned with one another. The compartment may contain first and second elastomeric elements therein, as described with respect to the housing 550 of the catheter securement device 500. For example, a first elastomeric element, such as 550 described above, may be positioned within the compartment 662 and that contains a passageway configured to receive and secure a catheter tube therein. The passageway extends from the first opening 663 in the housing front wall 660a to the second opening 665 in the housing rear wall 660b. The compartment and catheter tube extending therethrough can then be enclosed by a cover 690.

As illustrated in FIG. 38, the compartment 662 is also configured to receive a catheter hub CH therewithin, for example, after removal of the elastomeric element (i.e., the first elastomeric element 560 of FIG. 31) for securing a catheter tube.

Similar to the catheter securement device 500, the housing 660 is configured to be adjustably movable on the base 610 via first and second guide rails 680, 682. The first section 612 includes the first guide rail 680 on the first surface 612e thereof, and the second section 614 includes the second guide rail 682 on the first surface thereof 614e. The bottom surface of the housing includes a first feature, such as spaced apart walls, configured to slidingly engage the first guide rail 680 and a second feature, such as spaced apart walls, configured to slidingly engage the second guide rail 682 such that the housing 660 is movable relative to the base 610. A cover 690 is pivotably secured to the housing 660 via a hinge assembly 692 and is movable between an open position exposing the compartment 662 and a closed position covering the compartment 662. The cover includes a latch mechanism 694 configured to releasably engage with a retaining feature 622 in a similar manner as described above with respect to the catheter securement device 500.

When the cover 690 is in the closed position, the cover 690 provides a downward force on the housing 650 that is sufficient to restrict movement of the housing 650 relative to the base 610. When the cover 690 is moved to an open position, the housing 650 can be moved relative to the base 610.

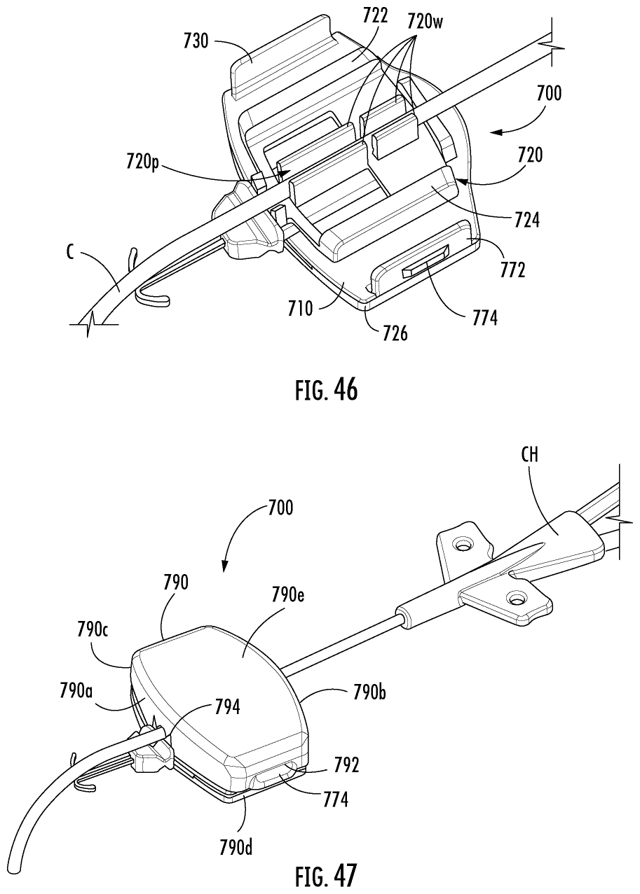

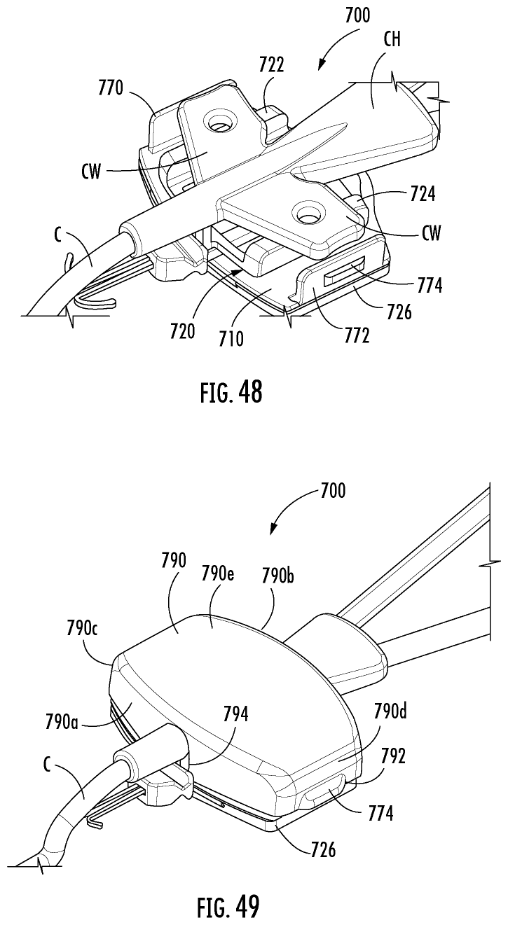

Referring to FIGS. 43-49, a catheter securement device 700 according to some embodiments of the present invention is illustrated. The illustrated catheter securement device 700 includes a base 710 having opposite front and rear edges 710a, 710b, opposite side edges 710c, 710d, a first surface 710e, and an opposite second surface 710f. A first elastomeric element 720 is secured to the base first surface 710e and includes a plurality of spaced apart elastomeric walls 720w extending outwardly therefrom. The plurality of spaced apart walls define a passageway 720p configured to receive and secure a catheter tube therein (FIG. 46). In addition, the plurality of spaced apart walls are configured to deform and support a catheter hub thereon (FIG. 48).

The first elastomeric element 720 also includes spaced apart side walls 722, 724, which are configured to support the wings CW of a catheter hub CH when placed on the first elastomeric element 720. The body of the catheter hub CH deforms the walls 720 in the medial portion of the elastomeric element 720 such that the wings CW of the catheter hub CH rest upon the walls 722, 724, as illustrated in FIG. 48. A second elastomeric element 726 is secured to the second surface 710f of the base 710. The second elastomeric element 726 is configured to contact the skin of a patient when the catheter securement device 700 is attached to the patient. The second elastomeric element 726 is configured to be comfortable to a patient.

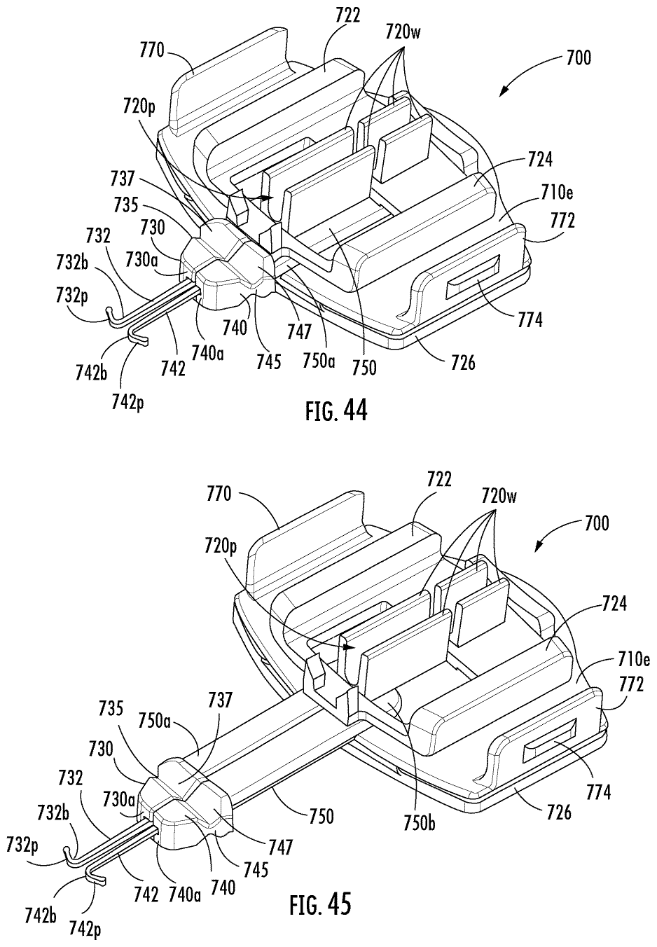

The illustrated catheter securement device 700 includes an elongate member 750 that is slidably secured to the base 710 and is movable between a retracted position (FIG. 44) and an extended position (FIG. 45). In the retracted position, a distal end 750a of the elongate member 750 is adjacent the base front edge 710a. In the extended position, the distal end 750 of the elongate member 750 is spaced apart from the base front edge 710a. A first flexible hinge member 730 is secured to the distal end 750a of the elongate member 750, and a second flexible hinge member 740 is secured to the distal end 750a of the elongate member 750 adjacent the first flexible hinge 730. The first flexible hinge member 730 includes a first arm 732 extending outward from a front end 730a of the first hinge member 730. The first arm 732 includes a first resilient prong 732p at a distal end 732b thereof. The second flexible hinge member 740 includes a second arm 742 extending outward from a front end 740a of the second hinge member 740. The second arm 740 includes a resilient prong 742p at a distal end 742b thereof. In some embodiments, the first arm 732 and first prong 732p and the second arm 742 and second prong 742p are substantially co-planar when in their respective relaxed states.

Similar to the catheter securement devices 500, 600 described above, the first and second flexible hinge members 730, 740 are configured to allow the base 710 supporting a catheter tube or catheter hub to pivot upwardly away from the skin of a patient while maintaining the prongs 732p, 742p of the first and second arms 732, 742 subcutaneously anchored in the skin of the patient, in a similar manner as illustrated above with respect to the embodiment in FIG. 37. This allows for cleaning around the insertion site of a catheter supported by the device 700. In the illustrated embodiment, a medial portion 735 of the first flexible hinge member 730 has a V-shaped notch 737 that defines a flexure region and, similarly, a medial portion 745 of the second flexible hinge member 740 has a V-shaped notch 747 that defines a flexure region. Each notch 737, 747 lowers the bending stiffness of the respective hinge member 730, 740 such that the hinge members 730, 740 bend easily at their respective medial portions 735, 745.

Similar to the catheter securement device 600 described above, an enclosure 760 is removably secured to the first and second flexible hinge members 730, 740 prior to attachment of the catheter securement device 700 to a patient. The illustrated enclosure 760 includes a first portion 762 that snugly fits over the first and second flexible hinge members 730, 740 from above, and a second portion 764 that extends outwardly from the first portion 762. In the illustrated embodiment, the second portion 764 is an elongate tube with an open distal end 764a and a slot 766 formed therein that extends from the open distal end 764a along the entire length of the tube, as illustrated. The second portion 764 is configured to retain the first arm 732 and first prong 732p in an unrelaxed, generally straight configuration, and to retain the second arm 742 and second prong 742p in an unrelaxed, generally straight configuration to facilitate insertion of the first and second prongs 732p, 742p through an incision in the skin of a patient. The second portion 764 has an atraumatic profile so that it can pass through an incision in a patient with minimal risk of snagging, lacerating, or otherwise damaging patient tissue.

When attaching the catheter securement device 700 to a patient, the second portion 764 is inserted into an incision in a patient, and the enclosure 760 is then removed from the device 700 by a user, such as a healthcare provider/technician, caretaker, etc. The user lifts up on the enclosure 760 to remove the first portion 762 from the first and second flexible hinge members 730, 740 and the second portion 764 from surrounding the first and second arms 732, 742 and respective prongs 732p, 742p. This causes the first prong 732p of the first arm 732 to return to a relaxed state extending laterally from the first arm 732 in a first direction, and the second prong 742p of the second arm 742 to return to a relaxed state extending laterally from the second arm 742 in a second direction different from the first direction. As a result, the first and second prongs 732p, 742p become subcutaneously anchored in the skin of the patient.

The first and second flexible hinge members 730, 740 are configured to allow the base 710 to pivot upwardly away from the skin while maintaining the first and second prongs 732p, 742p subcutaneously anchored in the skin of the patient, as described above. The ability to extend the elongate member 750 from a retracted position allows a user to adjust how far the base 710 is moved away from the skin of a patient, which can further facilitate cleaning around a catheter insertion site.

The illustrated catheter securement device 700 also includes a cover 790 that can be removably secured to the base 710 to facilitate the securing of a catheter or catheter hub to the base 710. The cover 790 is shaped and configured to overlie the base 710 with a catheter and/or catheter hub secured to the base 710. The cover 790 includes opposite front and rear walls 790a, 790b, opposite side walls 790c, 790d, and a top wall 790e. Each of the side walls 790c, 790d of the cover 790 includes a slot 792 formed therethrough. The base 710 includes a pair of opposing walls 770, 772 for securing the cover 790 to the base 710. One wall 770 extends upwardly from the first surface 710e adjacent the side edge 710c, and the other wall 772 extends upwardly from the first surface 710e adjacent the side edge 710d, as illustrated. Each wall 770, 772 includes a tab 774 projecting outwardly from an outer surface of the wall 770, 772. The tab 774 extending from wall 770 is not visible.

When the cover 790 is seated over the base 710, the outwardly extending tabs 774 on the base side walls 770, 772 are received in and engage the corresponding slots 792 in the cover side walls 790c, 790d. In this manner, the engagement between the tabs 774 and the slots 792 releasably secures the cover 790 to the base 710 while still allowing the cover 790 to be manually removed from the base 710 by deflecting the base side walls 770, 772 and/or lifting the cover 790 so that the tabs 774 disengage from the slots 792.

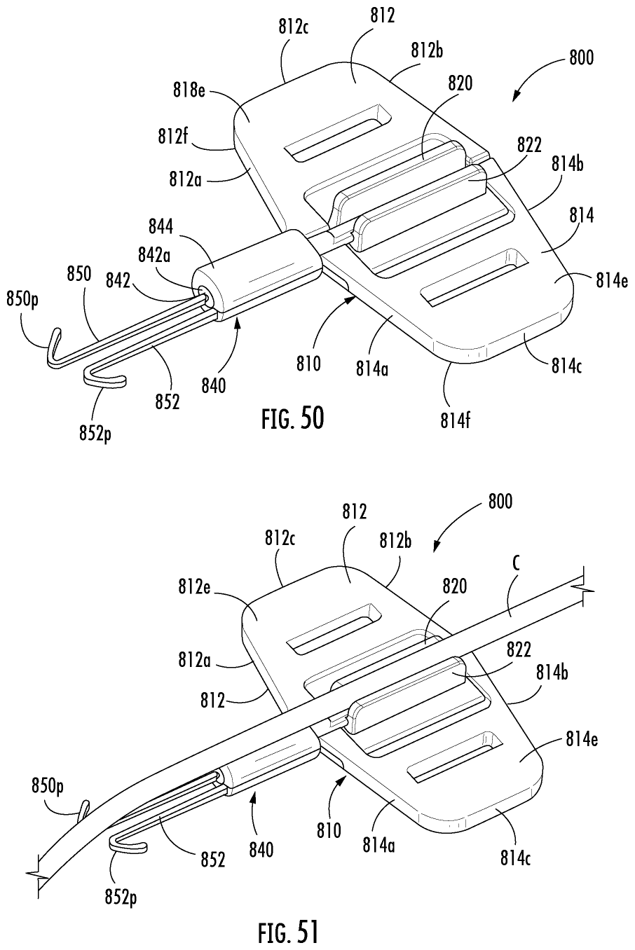

The front wall 790a of the cover 790 includes an opening 794 and the rear wall 790b includes a corresponding opening (not shown) that facilitate passage of a catheter C and/or a catheter hub CH therethrough, as illustrated in FIGS. 47 and 49.

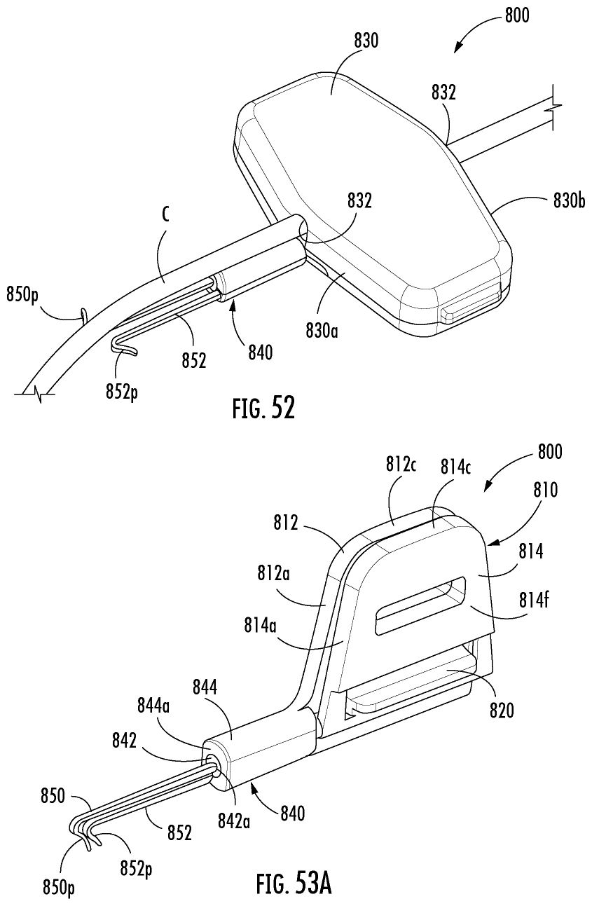

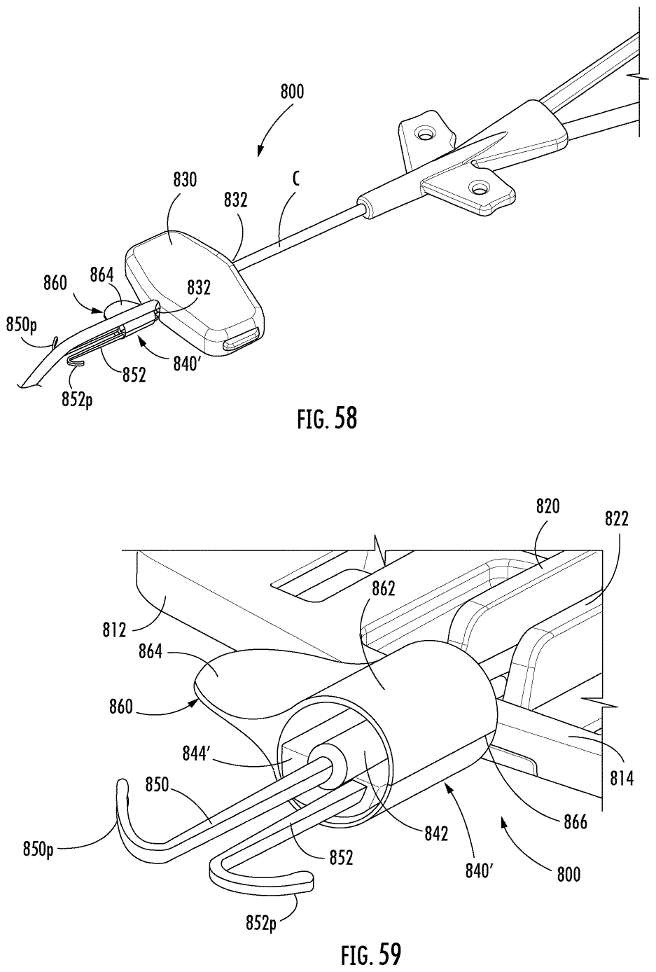

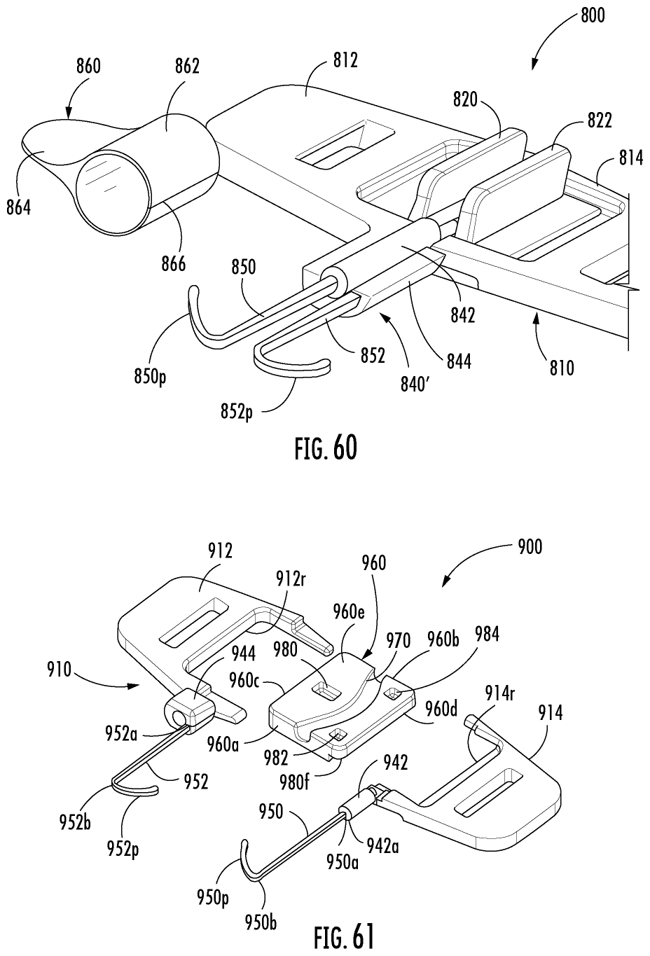

Referring to FIGS. 50-60, a catheter securement device 800 according to some embodiments of the present invention is illustrated. The illustrated catheter securement device 800 includes a base 810 having a first section 812 and a second section 814 movably coupled together. The first and second sections 812, 814 are movable between a first configuration wherein the first and second sections are substantially co-planar (FIGS. 50-52) and a second configuration wherein the first and second sections 812, 814 are in face-to-face relationship (FIG. 53A). The first and second sections 812, 814 each have a generally planar shape. The first section 812 includes opposite front and rear edges 812a, 812b, opposite side edges 812c, 812d, and opposite first and second surfaces 812e, 812f. The second section 814 includes opposite front and rear edges 814a, 814b, opposite side edges 814c, 814d, and opposite first and second surfaces 814e, 814f.

A first wall 820 extends outwardly from the first surface 812e of the first section 812 and a second wall 822 extends outwardly from the first surface 814e of the second section 814. The first and second walls 820, 822 are in adjacent spaced apart relationship and are configured to receive and secure a catheter tube C therein when the first and second sections are in a co-planar configuration (FIG. 51). The catheter securement device 800 also includes a cover 830 that is configured to be removably secured to the base 810 and overlie a catheter secured between the two walls 820, 822. The cover 830 has a shape that generally corresponds to the shape of the base 810 and includes openings 832 in the opposing front and rear walls 830a, 830b to facilitate passage therethrough of a catheter tube C secured between the first and second walls 820, 822.

The first and second sections 812, 814 are movably secured together by a hinge assembly 840 that includes a first hinge member 842 that is connected to the second section 814 and that and rotates in response to movement of the second section 814, and a second hinge member 844 that is connected to the first section 812 and that rotates in response to movement of the first section 812. FIG. 53B illustrates the second section 814 being rotated upwardly from the co-planar relationship of FIG. 50 and the corresponding rotation of the first arm 850 and prong 850p. FIG. 53C illustrates the first section 812 being rotated upwardly from the co-planar relationship of FIG. 50 and the corresponding rotation of the second arm 852 and prong 852p.

In the illustrated embodiment, the second hinge member 844 includes a longitudinal bore and the first hinge member 842 is a cylindrical pin positioned within the longitudinal bore such that the first and second hinge members 842, 844 rotate freely relative to each other. However, other hinge configurations may be utilized.

A first arm 850 extends outward from a front end 842a of the first hinge member 842 and includes a prong 850p at a distal end 850b of the first arm 850. An opposite proximal end 850a of the first arm 850 is secured to the first hinge member 842. A second arm 852 extends outward from a front end 844a of the second hinge 844 member and includes a prong 852p at a distal end 852b of the second arm 852. An opposite proximal end 852a of the second arm 852 is secured to the second hinge member 844. The first arm 850 is longer than the second arm 852 such that, when the first and second sections 812, 814 are moved to the configuration wherein the first surfaces 812e, 814e of each are in face to face relationship (FIG. 53A), the first arm 850 and second arm 852 are aligned in the same plane (i.e., the first arm 850 and second arm 852 are in adjacent, overlapping relationship) as illustrated in FIG. 53A, and the prongs 850p, 852p extend in substantially the same direction. The orientation of the arms 850, 852 when the first and second sections 812, 814 are in the configuration illustrated in FIG. 53A facilitates the insertion and removal of the prongs 850p, 852p through an incision in the skin of a patient. Thus, when a user is attaching the catheter securement device 800 to a patient, the first and second sections 812, 814 of the base 810 are folded together as illustrated in FIG. 53A and the prongs 850p, 852p are inserted through an incision in the patient. Upon insertion through the incision, the user then moves the first and second sections 812, 814 of the base 810 to the coplanar configuration to cause the prongs 850p, 852p of the first and second arms 850,852 to become subcutaneously anchored in the skin of the patient. To subsequently remove the catheter securement device 800 from the patient, the first and second sections 812, 814 of the base 810 are folded together as illustrated in FIG. 53A to facilitate removal of the prongs 850p, 852p from the patient.

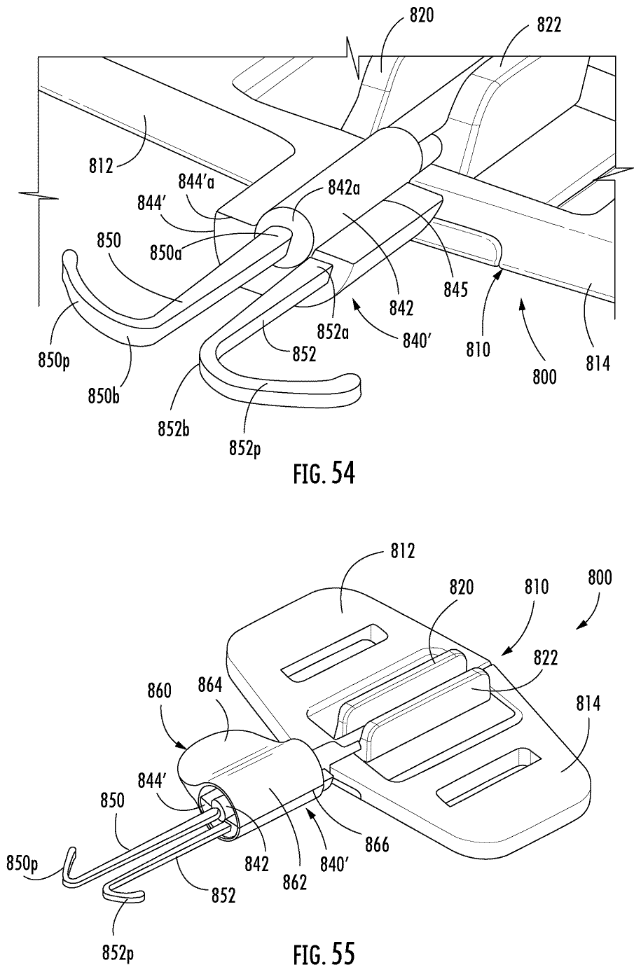

Referring to FIGS. 54-60, an alternative hinge assembly 840′ for the catheter securement device 800 of FIG. 50 is illustrated. The second hinge member 844′ has a semicylindrical configuration with a longitudinal channel 845. The first hinge member 842 is a cylindrical pin, as in the embodiment of FIG. 50, and is rotatably positioned within the longitudinal channel 845 of the second hinge member 844′. The first and second hinge members 842, 844′ are freely rotatable relative to each other. A retaining member 860 surrounds the first and second hinge members 842, 844′ and is configured to prevent the first hinge member 842 from dislodging from the longitudinal channel 845 of the second hinge member 844′ during use of the catheter securement device 800.

The illustrated retaining member 860 has a tubular shaped body 862 that is sized and configured to extend closely around the first and second hinge members 842, 844′ such that the first and second hinge members 842, 844′ can freely rotate relative to each other, but also not become dislodged from each other. The illustrated retaining member 860 also includes a handle 864 extending outwardly from one side of the body 862 and an elongated slit 866 formed in an opposite side of the body 862. The body 862 is formed from an elastomeric material such that a user can pull on the handle 864 and the body 862 expands such that the first and second hinge members 842, 844′ can pass through the slit 866. This feature allows a user to separate the first and second sections 812, 814 of the catheter securement device 800 so as to remove each section 812, 814 separately from a patient.

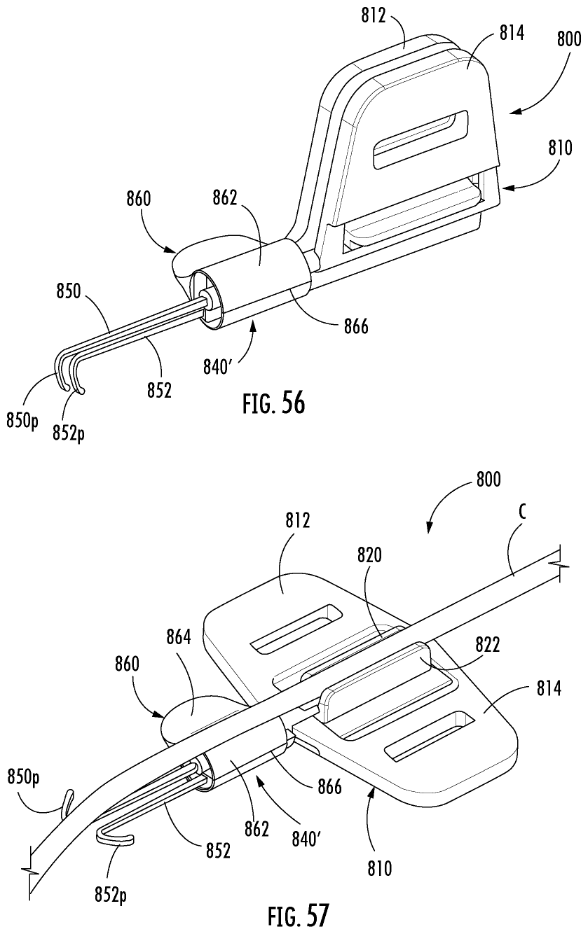

FIG. 56 illustrates the orientation of the first and second sections 812, 814 for insertion of the prongs 850p, 852p through an incision in a patient. However, the ability to separate the first and second sections 812, 814 by removing the retaining member 860 such that the first and second sections 812, 814 can be removed separately provides an alternative and potentially less painful way of removing the catheter securement device 800 from a patient.

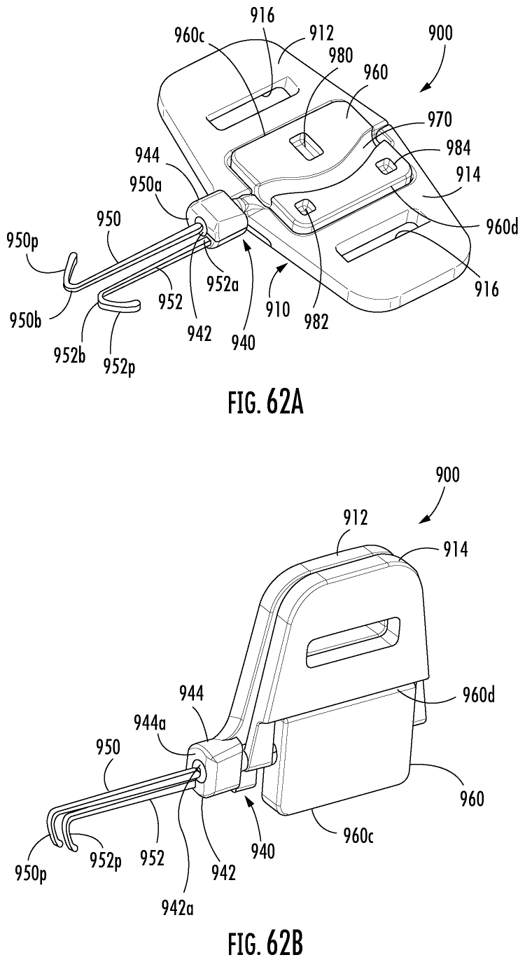

Referring to FIGS. 61-63, a catheter securement device 900 according to some embodiments of the present invention is illustrated. The illustrated catheter securement device 900 includes a base 910 having a first section 912 and a second section 914 movably coupled together by a hinge assembly 940. The hinge assembly 940 includes a first hinge member 942 attached to the second section 914, and a second hinge member 944 attached to the first section 912. The first hinge member 942 and the second hinge member 944 are operably connected such that the first and second sections 912, 914 are movable between a first configuration where the first and second sections 912, 914 are substantially co-planar (FIG. 62A) and a second configuration where the first and second sections 912, 914 are folded together and in face-to-face relationship (FIG. 62B).

A first arm 950 extends outward from a front end 942a of the first hinge member 942 and includes a prong 950p at a distal end 950b of the first arm 950 thereof. The opposite proximal end 950a of the first arm 950 is secured to the first hinge member 942. A second arm 952 extends outward from a front end 944a of the second hinge member 944 and includes a prong 952p at a distal end 952b of the second arm 952. The opposite proximal end 952a of the second arm 952 is secured to the second hinge member 944.

Similar to the catheter securement device 800, the first arm 950 of the catheter securement device 900 is longer than the second arm 952 such that, when the first and second sections 912, 914 are moved to the folded face-to-face configuration (FIG. 62B), the first arm 950 and second arm 952 are aligned in the same plane (i.e., the first arm 950 and second arm 952 are in adjacent, overlapping relationship) as illustrated in FIG. 62B, and the prongs 950p, 952p extend in substantially the same direction to facilitate the insertion and removal of the prongs 950p, 952p through an incision in the skin of a patient. Upon insertion through the incision in the skin of the patient and subsequently moving the first and second sections 912, 914 to the co-planar configuration, the prongs 950p, 952p of the first and second arms 950, 952 become subcutaneously anchored in the skin of the patient.

The illustrated catheter securement device 900 also includes an elastomeric member 960 that is removably attached to the second section 914 of the base 910. The second section 914 includes a recessed portion 914r within which the elastomeric member 960 is secured. The first section 912 includes a recessed portion 912r in which the elastomeric member 960 is positioned when the first and second sections 912, 914 are generally co-planar. When the first and second sections 912, 914 are folded to a face-to-face configuration as illustrated in FIG. 62B, the elastomeric member pivots with the second section and out of the recessed portion 912r of the first section 912.

The illustrated elastomeric member 960 has a generally rectangular shape with opposite front and rear edges 960a, 960b, opposite first and second side edges 960c, 960d, and opposite first and second surfaces 960 e, 960 f. The elastomeric member 960 second side edge 960d is secured to the second section recessed portion 914r when the catheter securement device 900 is assembled. The first side edge 960c is not secured to the first section 912 so that, when the first and second sections 912, 914 are folded together, the elastomeric member 960 and second section 914 are in face-to-face relationship with the first section 912, as illustrated in FIG. 62B.

A channel or passageway 970 is formed in the first surface 960e of the elastomeric member 960 and is configured to snugly receive a catheter tube inserted into a patient and prevent inadvertent movement of the catheter at the insertion site. The illustrated passageway 970 has an arcuate shape, but may have various configurations and is not limited to the illustrated configuration. For example, in some embodiments, the passageway 970 may be generally straight.

The illustrated elastomeric member 960 includes a first recessed portion 980 formed therein adjacent to the passageway 970 and between the passageway 970 and the first side edge 960c. The illustrated elastomeric member 960 also includes a second recessed portion 982 and a third recessed portion 984 formed therein adjacent to the passageway 970 and between the passageway 970 and the second side edge 960d.

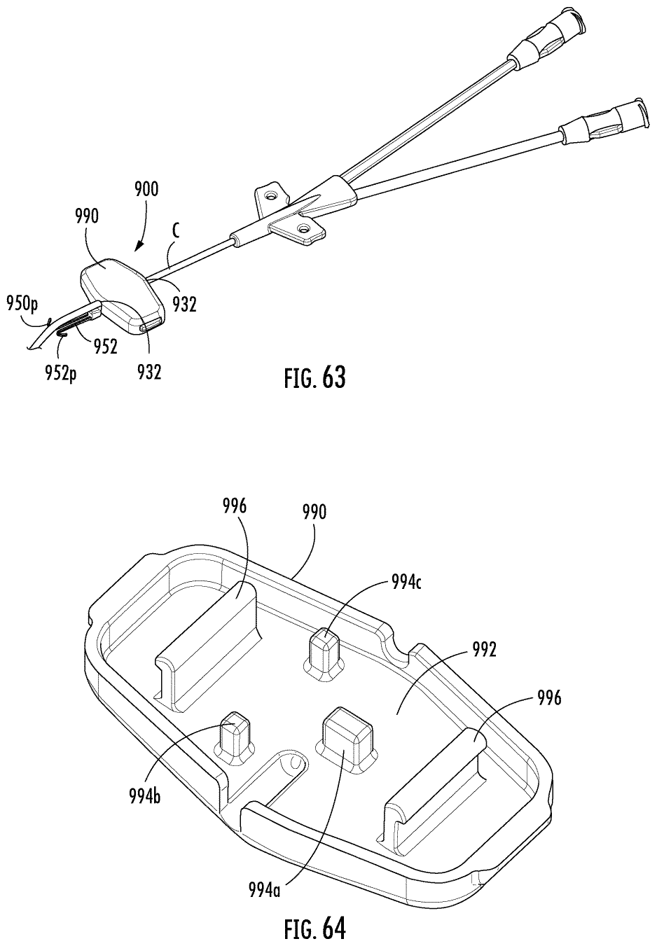

A cover 990 is configured to be secured to the base 910, as illustrated in FIG. 63. As illustrated in FIG. 64, the cover 990 includes first, second and third protrusions 994a, 994b, 994c extending outward from the cover inside surface 992. When the cover 990 is secured to the base 910 via members 996 engaging with respective slots 916 in the first and second sections 912, 914, the first protrusion 994a engages the first recessed portion 980, the second protrusion 994b engages the second recessed portion 982, and the third protrusion 994c engages the third recessed portion 984. The engagement of the first, second and third protrusions 994a, 994b, 994c with the respective first, second and third recessed portions 980, 982, 984 squeezes the passageway 970 and further helps prevent any unintended movement of a catheter tube C received in the passageway. Various configurations and numbers of protrusions and recessed portions may be utilized, and embodiments of the present invention are not limited to the illustrated number and configuration of the protrusions and recessed portions in the elastomeric member 960.

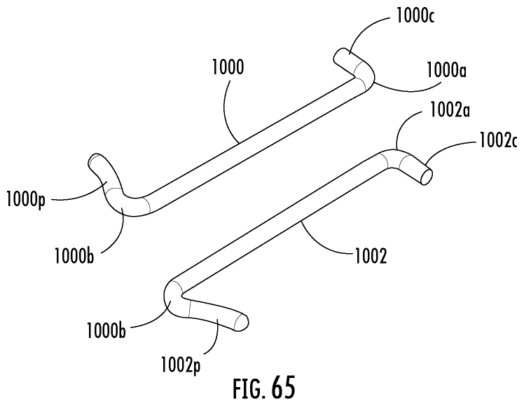

FIG. 65 is a perspective view of a pair of first and second arms 1000, 1002 that can be utilized with the various catheter securement devices described and illustrated above. The first arm 1000 includes opposite proximal and distal end portions 1000a, 1000b. A prong 1000p extends laterally from the first arm 1000 and has an atraumatic profile so that it can pass through an incision in a patient with minimal risk of snagging, lacerating, or otherwise damaging patient tissue. The distal end portion 1000a includes a member 1000c extending laterally from the first arm 1000 that is configured to be secured to a base or hinge assembly of a catheter securement device. Similarly, the second arm 1002 includes opposite proximal and distal end portions 1002a, 1002b. A prong 1002p extends laterally from the second arm 1002 and has an atraumatic profile so that it can pass through an incision in a patient with minimal risk of snagging, lacerating, or otherwise damaging patient tissue. The distal end portion 1002a includes a member 1002c extending laterally from the second arm 1002 that is configured to be secured to a base or hinge assembly of a catheter securement device.

The various catheter securement devices described herein may be formed from various materials including, but not limited to, all types of plastic materials, ceramic materials, metals, elastomeric materials, or combinations thereof.

Unless otherwise defined, all terms (including technical and scientific terms) used herein have the same meaning as commonly understood by one of ordinary skill in the art to which the present invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity. The term “and/or” includes any and all combinations of one or more of the associated listed items.

The term “relaxed state”, as used herein, means that an elastomeric or resilient element or member is substantially free of an externally applied load and substantially at equilibrium shape. The term “unrelaxed state”, as used herein, means that an elastomeric or resilient element or member is not yet at equilibrium and is still under load and has not yet fully recovered its equilibrium or relaxed shape.

Like numbers refer to like elements throughout. In the figures, certain components or features may be exaggerated for clarity, and broken lines may illustrate optional features or operations unless specified otherwise. In addition, the sequence of operations (or steps) is not limited to the order presented in the figures and/or claims unless specifically indicated otherwise. Features described with respect to one figure or embodiment can be associated with another embodiment or figure although not specifically described or shown as such.

It will be understood that when an element is referred to as being “on” another element, it can be directly on the other element or intervening elements may also be present. In contrast, when an element is referred to as being “directly on” another element, there are no intervening elements present. It will also be understood that when an element is referred to as being “connected”, “coupled”, “responsive”, or variants thereof to another element, it can be directly connected, coupled or responsive to the other element or intervening elements may be present. In contrast, when an element is referred to as being “directly connected”, “directly coupled”, “directly responsive”, or variants thereof to another element, there are no intervening elements present. Other words used to describe the relationship between elements should be interpreted in a like fashion (i.e., “between” versus “directly between”, “adjacent” versus “directly adjacent”, etc.).

As used herein, the singular forms “a”, “an” and “the” are intended to include the plural forms as well, unless the context clearly indicates otherwise. As used herein, the terms “comprise”, “comprising”, “comprises”, “include”, “including”, “includes”, “have”, “has”, “having”, or variants thereof are open-ended, and include one or more stated features, integers, elements, steps, components or functions but does not preclude the presence or addition of one or more other features, integers, elements, steps, components, functions or groups thereof. Furthermore, as used herein, the common abbreviation “e.g.,” which derives from the Latin phrase “exempli gratia,” may be used to introduce or specify a general example or examples of a previously mentioned item, and is not intended to be limiting of such item. The common abbreviation “i.e.,” which derives from the Latin phrase “id est,” may be used to specify a particular item from a more general recitation.

It will be understood that although the terms first, second, third, etc., may be used herein to describe various elements/operations, these elements/operations should not be limited by these terms. These terms are only used to distinguish one element/operation from another element/operation. Thus, a first element/operation in some embodiments could be termed a second element/operation in other embodiments without departing from the teachings of the present invention. The same reference numerals or the same reference designators denote the same or similar elements throughout the specification.

Relative terms such as “below” or “above” or “upper” or “lower” or “horizontal” or “vertical” may be used herein to describe a relationship of one element, layer or region to another element, layer or region as illustrated in the figures. It will be understood that these terms are intended to encompass different orientations of the device in addition to the orientation depicted in the figures.

The terms “about” and “approximately”, as used herein with respect to a value or number, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the teachings and advantages of this invention. Accordingly, all such modifications are intended to be included within the scope of this invention as defined in the claims. The invention is defined by the following claims, with equivalents of the claims to be included therein.

1-14. (canceled)

15. A catheter securement device, comprising:
a flexible base defining a compartment, the base comprising first and second portions, each having opposite first and second surfaces and opposite first and second ends, wherein the first and second portions are substantially co-planar when in a relaxed state, wherein the first and second portions are foldable along a centerline of the base to an unrelaxed state, wherein the second surface of the first portion and the second surface of the second portion are in face-to-face relationship when the first and second portions are folded to the unrelaxed state;
a first arm extending outward from the first end of the first portion, the first arm comprising a prong extending laterally from a distal end thereof; and
a second arm extending outward from the first end of the second portion, the second arm comprising a prong extending laterally from a distal end thereof,
wherein, when the first and second portions are in the relaxed state, the prong of the first arm extends laterally in a first direction and the prong of the second arm extends laterally in a second direction that is opposite from the first direction, and
wherein, when the first and second portions are folded to the unrelaxed state, the prong of the first arm and the prong of the second arm are in adjacent relationship and each extends in substantially a same direction to facilitate insertion and removal of the prong of the first arm and the prong of the second arm through an incision in the skin of a patient.

16. The catheter securement device of claim 15, wherein, upon insertion through the incision in the skin of the patient and allowing the first and second portions to return to the relaxed state, the prongs of the first and second arms become subcutaneously anchored in the skin of the patient.

17. The catheter securement device of claim 15, wherein the prong of the first arm and the prong of the second arm are substantially co-planar when the first and second portions are in the relaxed state.

18. The catheter securement device of claim 15, wherein the first portion comprises a first elongate slot and a first guide post slidably engaged therewithin, wherein the second portion comprises a second elongate slot and a second guide post slidably engaged therewithin, wherein the first and second posts extend outwardly from the respective first and second portions and are each configured to engage a respective aperture in a wing of a catheter hub placed within the compartment and such that the catheter hub is adjustably movable within the compartment, and wherein one of the first or second slots comprises teeth configured to engage and lock in place the respective post slidably engaged therewith.

19. (canceled)

20. The catheter securement device of claim 18, further comprising a cover pivotably secured to the base and movable between an open position exposing the compartment and a closed position covering the compartment.

21. A catheter securement device, comprising:
a base comprising:
a first section having an inner edge, an opposite outer edge, a front edge, an opposite rear edge, a first surface, and an opposite second surface;
a second section having an inner edge, an opposite outer edge, a front edge, an opposite rear edge, a first surface, and an opposite second surface; and
an elastomeric member removably secured to the first section inner edge and to the second section inner edge;
wherein the first and second sections are substantially co-planar when the elastomeric member is in a relaxed state, wherein the elastomeric member is foldable along a centerline thereof to an unrelaxed state, wherein the second surface of the first section and the second surface of the second section are in face-to-face relationship when the elastomeric member is folded to the unrelaxed state;
a first flexible hinge member secured to a portion of the first section front edge, the first flexible hinge member comprising a first arm extending outward from a front end of the first hinge member, the first arm comprising a prong extending laterally from a distal end thereof; and
a second flexible hinge member secured to a portion of the second section front edge, the second flexible hinge member comprising a second arm extending outward from a front end of the second hinge member, the second arm comprising a prong extending laterally from a distal end thereof,
wherein, when the elastomeric member is in the relaxed state, the prong of the first arm extends laterally in a first direction and the prong of the second arm extends laterally in a second direction that is opposite from the first direction, and
wherein, when the elastomeric member is folded to the unrelaxed state, the prong of the first arm and the prong of the second arm are in adjacent relationship and each extends in substantially a same direction to facilitate insertion and removal of the first and second prongs through an incision in the skin of a patient.

22. The catheter securement device of claim 21, wherein the elastomeric member comprises a first slot formed in a first side thereof that is configured to slidably retain the inner edge of the first section therein, and wherein the elastomeric member comprises a second slot formed in an opposite second side thereof that is configured to slidably retain the inner edge of the second section therein.

23. The catheter securement device of claim 21, wherein, upon insertion through the incision in the skin of the patient and subsequently allowing the elastomeric member to return to the relaxed state, the prongs of the first and second arms become subcutaneously anchored in the skin of the patient.

24. The catheter securement device of claim 23, wherein the first and second flexible hinge members are configured to allow the base to pivot upwardly away from the skin while maintaining the prongs of the first and second arms subcutaneously anchored in the skin of the patient.

25. The catheter securement device of claim 21, wherein the prong of the first arm and the prong of the second arm are substantially co-planar when the elastomeric member is in the relaxed state.

26. The catheter securement device of claim 21, further comprising:
a housing removably secured to the base, wherein the housing comprises opposite front and rear walls, opposite side walls, and a floor that define a compartment, wherein the front wall includes a first opening, and the rear wall includes a second opening, the first and second openings being generally aligned with one another; and
an elastomeric element removably positioned within the compartment, wherein the elastomeric element comprises a passageway extending from the first opening in the housing front wall to the second opening in the housing rear wall, and wherein the passageway is configured to receive and secure a catheter tube therein.

27. The catheter securement device of claim 26, wherein, when the elastomeric element is removed from the compartment, the compartment is configured to receive a catheter hub therewithin.

28. The catheter securement device of claim 26, further comprising:
a first guide rail on the first surface of the first section and a second guide rail on the first surface of the second section, wherein a bottom surface of the housing comprises a first feature configured to slidingly engage the first guide rail and a second feature configured to slidingly engage the second guide rail such that the housing is movable relative to the base; and
a cover pivotably secured to the housing and movable between an open position exposing the compartment and a closed position covering the compartment, and wherein movement of the housing relative to the base is restricted when the cover is in the closed position.

29. (canceled)

30. A catheter securement device, comprising:
a base comprising:
a first section having an inner edge, an opposite outer edge, a front edge, an opposite rear edge, a first surface, and an opposite second surface;
a second section having an inner edge, an opposite outer edge, a front edge, an opposite rear edge, a first surface, and an opposite second surface; and
wherein the first section inner edge abuts the second section inner edge and the first section and second section are maintained in a substantially co-planar configuration by a locking mechanism that removably engages the first section and the second section;
a first flexible hinge member secured to a portion of the first section front edge, the first flexible hinge member comprising a first arm extending outward from a front end of the first hinge member, the first arm comprising a first resilient prong at a distal end thereof;
a second flexible hinge member secured to a portion of the second section front edge, the second flexible hinge member comprising a second arm extending outward from a front end of the second hinge member, the second arm comprising a second resilient prong at a distal end thereof; and
an enclosure removably secured to the first and second flexible hinge members, wherein the enclosure comprises an elongate tube configured to retain the first arm and first prong in an unrelaxed configuration, and to retain the second arm and second prong in an unrelaxed configuration to facilitate insertion of the first and second prongs through an incision in the skin of a patient, and wherein, upon removal of the enclosure, the first prong is configured to return to a relaxed state extending laterally from the first arm in a first direction, and the second prong is configured to return to a relaxed state extending laterally from the second arm in a second direction different from the first direction.

31. The catheter securement device of claim 30, wherein, upon insertion through the incision in the skin of the patient and subsequent removal of the enclosure, the first and second prongs become subcutaneously anchored in the skin of the patient.

32. The catheter securement device of claim 31, wherein the first and second flexible hinge members are configured to allow the base to pivot upwardly away from the skin while maintaining the first and second prongs subcutaneously anchored in the skin of the patient.

33. The catheter securement device of claim 30, wherein the first prong and second prong are substantially co-planar when in their respective relaxed state.

34. The catheter securement device of claim 30, further comprising:
a housing removably secured to the base, wherein the housing comprises opposite front and rear walls, opposite side walls, and a floor that define a compartment, wherein the front wall includes a first opening, and the rear wall includes a second opening, the first and second openings being generally aligned with one another; and
an elastomeric element removably positioned within the compartment, wherein the elastomeric element comprises a passageway extending from the first opening in the housing front wall to the second opening in the housing rear wall, and wherein the passageway is configured to receive and secure a catheter tube therein.

35. The device of claim 34, wherein, when the elastomeric element is removed from the compartment, the compartment is configured to receive a catheter hub therewithin.

36. The catheter securement device of claim 34, further comprising:
a first guide rail on the first surface of the first section and a second guide rail on the first surface of the second section, wherein a bottom surface of the housing comprises a first feature configured to slidingly engage the first guide rail and a second feature configured to slidingly engage the second guide rail such that the housing is movable relative to the base; and
a cover pivotably secured to the housing and movable between an open position exposing the compartment and a closed position covering the compartment, and wherein movement of the housing relative to the base is restricted when the cover is in the closed position.

37-56. (canceled)
